(19) United States
(12) Patent Application Publication
(10) Pub. No.: US 2023/0329876 A1
(43) Pub. Date: Oct. 19, 2023

(54) MECHANICAL LIGAMENT BALANCING DEVICES, KITS, AND METHODS

(71) Applicant: Exactech, Inc., Gainesville, FL (US)

(72) Inventors: Laurent Angibaud, Gainesville, FL (US); Michael Mauldin, Gainesville, FL (US); Matt Rueff, Gainesville, FL (US); Noah Davis, Gainesville, FL (US)

(21) Appl. No.: 18/300,840

(22) Filed: Apr. 14, 2023

Related U.S. Application Data
(60) Provisional application No. 63/387,621, filed on Dec. 15, 2022.
(60) Provisional application No. 63/331,418, filed on Apr. 15, 2022.

(51) Int. Cl. A61F 2/44

(52) U.S. Cl. CPC A61F 2/442; A61F 2002/30579

(57) ABSTRACT

A device including first plate configured to interface with a first bone structure of a joint; a second plate configured to interface with a second bone structure of the joint; and at least one mechanical actuation mechanism disposed between the first plate and the second plate and configured to apply a distraction force so as to urge the first and second plates away from one another, wherein the at least one mechanical actuation mechanism includes first and second actuation sub-mechanisms configured to provide first actuation and second sub-mechanism distraction forces that are antagonist to one another; wherein the device has a range of expansion ranging from a minimum distance to a maximum distance between the first plate and the second plate, and wherein the first actuation sub-mechanism distraction force and the second actuation sub-mechanism distraction force combine to provide the distraction force that is substantially constant.

### CROSS-REFERENCE TO RELATED APPLICATIONS

This application is a Section 111(a) application relating to and claiming the benefit of commonly-owned, co-pending U.S. Provisional Pat. Application No. 63/331,418, filed on Apr. 15, 2022 and entitled “MECHANICAL LIGAMENT BALANCING DEVICES, KITS, AND METHODS,” and U.S. Provisional Pat. Application No. 63/387,621, filed on Dec. 15, 2022 and entitled “MECHANICAL LIGAMENT BALANCING DEVICES, KITS, AND METHODS,” the contents of both of which are incorporated herein by reference in their entirety.

### FIELD OF THE INVENTION

The field of invention relates to orthopedic surgery. More particularly, the field of invention relates to balancing devices that are used by surgeons to characterize a ligament and capsular envelope around a joint during surgery and to apply a tension to the ligament and capsular envelope around a joint during the same surgery.

### BACKGROUND OF THE INVENTION

Expandable ligament balancing devices are used to help the surgeon to assess the proper tension of the ligament envelope surrounding the joint at the time of the surgery. FIG. 1 shows a conventional ligament balancing device. These devices (e.g., devices disclosed by U.S. Pat. No. 10,154,836) include a proximal plate 100, a distal plate 200, and an expandable member 300 located between the proximal plate 100 and the distal plate 200. The expandable member 300 is controlled through an expansion mechanism using an electrical source, an electromechanical source, a mechanical source, a pneumatic source, a hydraulic source, or any combination of these sources.

### SUMMARY OF THE DISCLOSURE

In some embodiments, a device includes a first plate configured to interface with a first bone structure of a joint; a second plate configured to interface with a second bone structure of the joint opposite the first bone structure; and at least one mechanical actuation mechanism disposed between the first plate and the second plate and configured to apply a distraction force along an axis between the first plate and the second plate so as to urge the first plate and the second plate away from one another, wherein the at least one mechanical actuation mechanism includes: a first actuation sub-mechanism, and a second actuation sub-mechanism, wherein the first actuation sub-mechanism is configured to provide a first actuation sub-mechanism distraction force, and wherein the second actuation sub-mechanism is configured to provide a second actuation sub-mechanism distraction force that is antagonist to the first actuation sub-mechanism distraction force; wherein the device is configured so as to have a range of expansion ranging from a minimum distance between the first plate and the second plate to a maximum distance between the first plate and the second plate, and wherein the first actuation sub-mechanism distraction force and the second actuation sub-mechanism distraction force combine to provide the distraction force that is a substantially constant distraction force across the range of expansion.

In some embodiments, the device is configured so as to allow the substantially constant distraction force to be adjusted. In some embodiments, the first actuation sub-mechanism includes an adjustment element that is one of adjustable or interchangeable to thereby adjust the substantially constant distraction force. In some embodiments, the adjustment element includes a spring having an adjustable preload. In some embodiments, the adjustment element includes an interchangeable cartridge. In some embodiments, the adjustment element includes an interchangeable spring. In some embodiments, the device is configured so as to allow the substantially constant distraction force to be adjusted while positioned in situ.

In some embodiments, the device is configured to apply the substantially constant distraction force to a single condyle of a bicondylar joint. In some embodiments, the device is configured to be joined to a further one of the device so as to apply the substantially constant distraction force to both condyles of a bicondylar joint.

In some embodiments, a kit includes a distraction device, including: a first fixation location configured to receive a first bone contacting element so as to position the first bone contacting element so as to be configured to interface with a first bone structure of a joint; a second fixation location configured to receive a second bone contacting element so as to position the second bone contacting element so as to be configured to interface with a second bone structure of the joint opposite the first bone structure; and a mechanical actuation mechanism disposed between the first fixation location and the second fixation location and configured to apply a distraction force along an axis between the first fixation location and the second fixation location so as to urge the first bone contacting element and the second bone contacting element away from one another, wherein the mechanical actuation mechanism includes a force applying element configured to apply an applied force so as to urge the first bone contacting element and the second bone contacting element away from one another, wherein a magnitude of the applied force varies as the device moves along a range of expansion, wherein the mechanical actuation mechanism includes a physical parameter that varies as the device moves along the range of expansion, and wherein the varying applied force and the varying physical parameter combine to cause the distraction force to be a substantially constant distraction force across the range of expansion; and a plurality of bone contacting elements, wherein the first bone contacting element and the second bone contacting element are selected from among the plurality of bone contacting elements.

In some embodiments, the device is configured so as to allow the substantially constant distraction force to be adjusted. In some embodiments, the varying physical parameter is a varying moment arm. In some embodiments, the device includes an adjustment dial that is adjustable so as to adjust the varying moment arm. In some embodiments, the adjustment dial is adjustable to a plurality of discrete settings.

In some embodiments, the distraction device is configured such that, when in use, the first bone contacting element and the second bone contacting element are positioned in an intraarticular position and the mechanical actuation mechanism is positioned in an extraarticular position.

In some embodiments, at least one of the plurality of bone contacting elements includes a trial.

In some embodiments, at least one of the plurality of bone contacting elements includes a tracker.

In some embodiments, at least one of the plurality of bone contacting elements is configured to be secured to bone.

In some embodiments, at least one of the plurality of bone contacting elements includes a textured surface.

In some embodiments, the first fixation location is configured to allow the first bone contacting element to rotate with respect to the first fixation location about an axis that is perpendicular to an axis of expansion of the distraction device.

In some embodiments, the distraction device includes a scale and a pointer, and the distraction device is configured such that the pointer indicates a current gap between the first bone contacting element and the second bone contacting element on the scale. In some embodiments, the distraction device includes a marker slidably positioned on the scale, and the pointer is configured to slide the marker along the scale such that the marker indicates one of (1) a maximum gap between the first bone contacting element and the second bone contacting element or (2) a minimum gap between the first bone contacting element and the second bone contacting element.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 shows a conventional ligament balancing device;

FIG. 2A shows the available range of tilt when the spacing (i.e., the height) of the ligament balancing device of FIG. 1 is above the threshold value;

FIG. 2B shows the available range of tilt when the spacing (i.e., the height) of the ligament balancing device of FIG. 1 is below the threshold value;

FIG. 2C shows the available range of tilt when the spacing (i.e., the height) of the ligament balancing device of FIG. 1 is at the high or low end of its spacing range;

FIG. 3 shows an alternate view of the conventional ligament balancing device of FIG. 1;

FIG. 4A shows a perspective view of an exemplary ligament balancing device;

FIG. 4B shows a partially transparent perspective view of the exemplary ligament balancing device of FIG. 4A;

FIG. 5A shows dimensions of an exemplary scissor mechanism;

FIG. 5B shows a side view of the exemplary ligament balancing device of FIG. 4A in a compressed position;

FIG. 5C shows a side view of the exemplary ligament balancing device of FIG. 4A in a partially expanded position;

FIG. 5D shows a side view of the exemplary ligament balancing device of FIG. 4A in a further expanded position;

FIG. 6A shows a kit including an exemplary ligament balancing device exoskeleton and exemplary cartridges;

FIG. 6B shows an exploded view of an exemplary cartridge;

FIG. 7A shows a selected exemplary cartridge;

FIG. 7B shows use of an insertion device to insert the selected exemplary cartridge of FIG. 7A into the exemplary exoskeleton of FIG. 6A;

FIG. 7C shows the exemplary exoskeleton of FIG. 6A with the selected exemplary cartridge of FIG. 7A inserted therein;

FIG. 8A shows a graph of distraction force against expansion for a first exemplary cartridge;

FIG. 8B shows a graph of distraction force against expansion for a second exemplary cartridge;

FIG. 8C shows a graph of distraction force against expansion for a third exemplary cartridge;

FIG. 9A shows an exemplary embodiment of a ligament balancing device;

FIG. 9B shows an exemplary embodiment of a ligament balancing device;

FIG. 10 shows an exemplary embodiment of a ligament balancing device;

FIG. 11A shows a perspective view of an exemplary embodiment of a ligament balancing device;

FIG. 11B shows a bottom view of the ligament balancing device of FIG. 11A;

FIG. 12A shows an exemplary embodiment of a ligament balancing device;

FIG. 12B shows use of an insertion device to insert a scissor leaf spring of an exemplary ligament balancing device;

FIG. 12C shows use of an insertion device to insert a leaf spring of an exemplary ligament balancing device;

FIG. 13 shows an exemplary embodiment of an extraarticularly-actuated ligament balancing device;

FIG. 14 shows an exemplary embodiment of a kit;

FIG. 15A shows a top view of an exemplary embodiment of a ligament balancing device;

FIG. 15B shows a perspective view of the ligament balancing device of FIG. 15A;

FIG. 16 shows an exemplary embodiment of a ligament balancing device;

FIG. 17A shows an exemplary embodiment of a unicondylar ligament balancing device;

FIG. 17B shows an exemplary embodiment of a unicondylar ligament balancing device;

FIG. 17C shows an exemplary embodiment of a bicondylar ligament balancing device formed from two of the unicondylar ligament balancing device of FIG. 17B;

FIG. 18 shows an exemplary kit;

FIG. 19A shows a top perspective view of an exemplary ligament balancing device;

FIG. 19B shows a bottom perspective view of the exemplary ligament balancing device of FIG. 19A;

FIG. 19C shows a bottom view of the exemplary ligament balancing device of FIG. 19A;

FIG. 19D shows a bar graph of distraction force against expansion for the exemplary ligament balancing device of FIG. 19A;

FIG. 19E shows a bar graph of distraction force against expansion for the exemplary ligament balancing device of FIG. 19A;

FIG. 19F shows a bar graph of distraction force against expansion for the exemplary ligament balancing device of FIG. 19A;

FIG. 20A shows a perspective view of an exemplary ligament balancing device;

FIG. 20B shows a bottom view of the exemplary ligament balancing device of FIG. 20A;

FIG. 21A shows an exemplary ligament balancing device and an exemplary compression handle;

FIG. 21B shows a workflow for an exemplary “tibia first” technique for total knee arthroplasty;

FIG. 22A shows a workflow for an exemplary “modified gap balancing” technique for total knee arthroplasty;

FIG. 22B shows an exemplary ligament balancing device and an exemplary spacer provided for use during a technique such as the technique shown in FIG. 22A; and

FIG. 23 shows an exemplary “full femur first” technique for total knee arthroplasty;

FIG. 24A shows a perspective view of an exemplary ligament balancing device;

FIG. 24B shows a partial side perspective view of the exemplary ligament balancing device of FIG. 24A;

FIG. 24C shows a partial side perspective view of the exemplary ligament balancing device of FIG. 24A;

FIG. 24D shows a partial side perspective view of the exemplary ligament balancing device of FIG. 24A;

FIG. 24E shows a partial side view of the exemplary ligament balancing device of FIG. 24A;

FIG. 24F shows a partial side view of the exemplary ligament balancing device of FIG. 24A;

FIG. 24G shows a partial side view of the exemplary ligament balancing device of FIG. 24A;

FIG. 24H shows the exemplary ligament balancing device of FIG. 24A as positioned within reference to a representative patient;

FIG. 24I shows a scale of the exemplary ligament balancing device 2400 of FIG. 24A;

FIG. 24J shows the exemplary ligament balancing device of FIG. 24A as positioned in a most compressed position;

FIG. 24K shows the exemplary ligament balancing of FIG. 24A as positioned in a most expanded position;

FIG. 25A shows an exemplary paddle having a concave contact surface;

FIG. 25B shows an exemplary paddle having a convex contact surface;

FIG. 25C shows exemplary paddles having flat contact surfaces;

FIG. 25D shows an exemplary paddle having a pin hole configured to receive a pin;

FIG. 25E shows exemplary paddles including fixture points that are configured to receive instruments from an implant kit;

FIG. 25F shows the exemplary ligament balancing device of FIG. 24A as configured with the exemplary paddles of FIG. 25E and having trial surgical instruments coupled thereto;

FIG. 25G shows exemplary paddles having trackers coupled thereto;

FIG. 25H shows an exemplary connection point providing a degree of rotational freedom to an attached paddle; and

FIG. 25I shows an exemplary embodiment of a kit.

### DETAILED DESCRIPTION

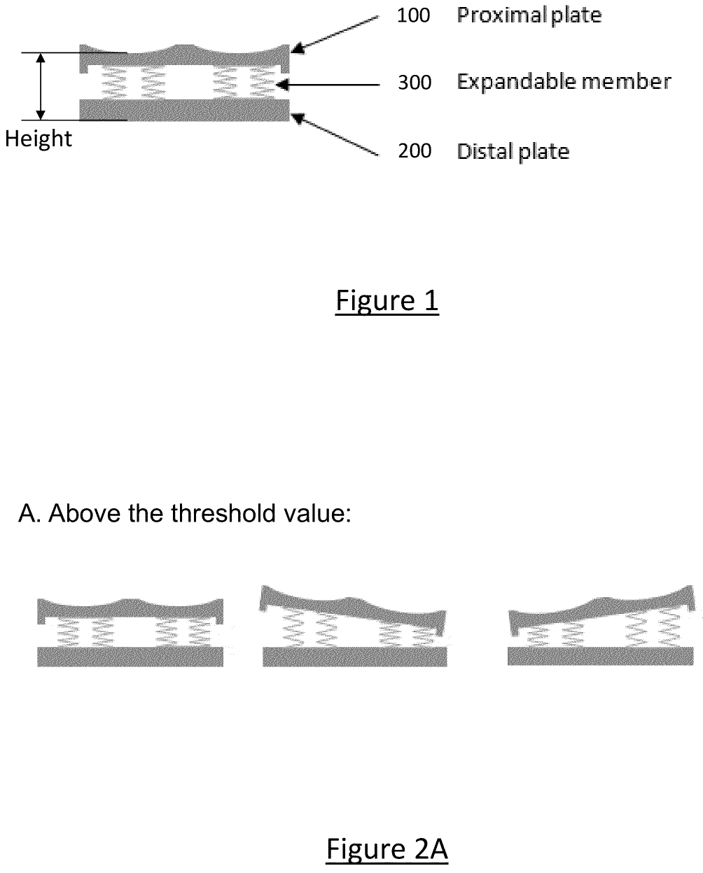

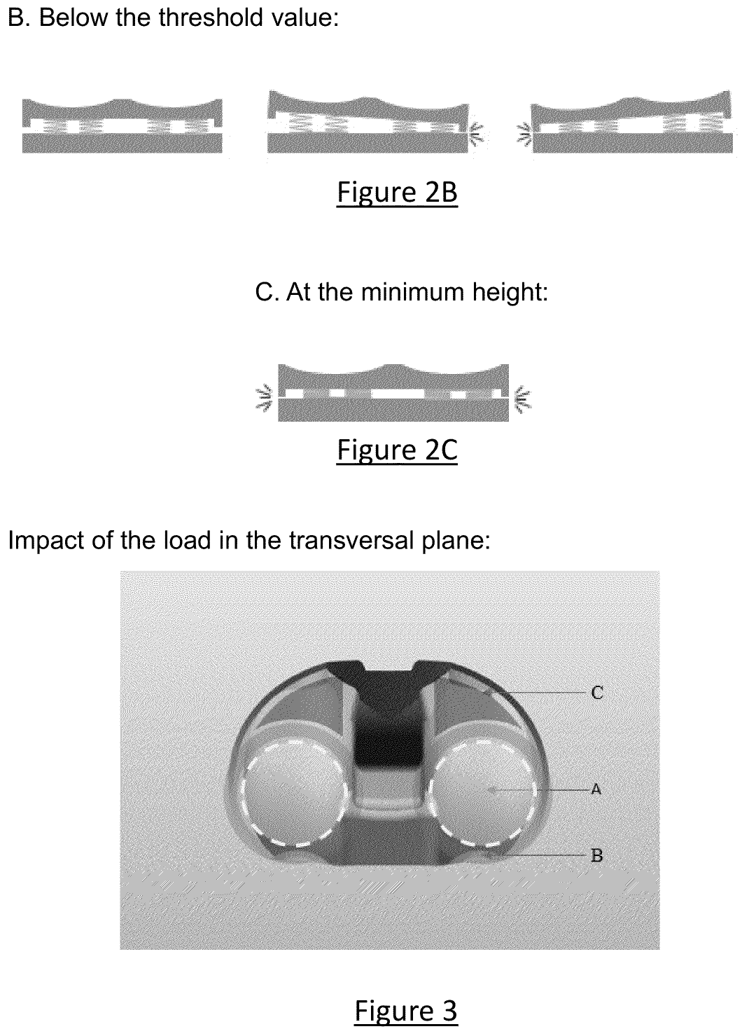

The inventors of the present application have identified certain limitations of conventional ligament balancing devices such as that shown in FIG. 1. A first limitation relates to the limited range of adjustment of the proximal plate 100 relative to the distal plate 200 in terms of height defined as the distance between the proximal plate 100 and the distal plate 200 (e.g., 8 to 14 mm), angular tilt defined as the sagittal and/or coronal orientation of the proximal plate 100 relative to the distal plate 200 (e.g., ±6°) and the interdependence between these two parameters. Based on the current architecture, the full range of angular tilt is only available when the height is above a threshold (e.g., at least between 10 and 12 mm), as shown in FIG. 2A. As the height is closer to its extreme minimum value, then the range of angular tilt is decreased. FIG. 2B shows a limited available range of angular tilt when the height of a conventional ligament balancing device on one side is below the threshold value. FIG. 2C shows the absence of available range of angular tilt when the height of a conventional ligament balancing device on one side is at its minimum (e.g., 8 mm). In some cases, this limitation may be significant for bi-compartmental types of joints (e.g., during total knee arthroplasty), in which one compartment may be substantially tighter than the other compartment.

A second limitation relates to the impact of the loading condition between the considered joint and the mobile plate (i.e., the proximal plate 100 or the distal plate 200 depending on the indication) in the transversal plane on the height and angular tilt measurements. There are two individual sources of error regarding this limitation. The first source of error relates to the location of the load relative to the expandable member 300. With reference to FIG. 3, which shows a profile view of a conventional ligament balancing device, the location of the load application impacts the measured gaps and/or angular tilts. For example, when the application of the load is directly located “inside” the expandable transversal cross-section (Area A), then the impact is negligible. Area B: When the application of the load is located on the most posterior aspect or the most lateral aspect of the articular surface (Area B), then the impact may not be clinically relevant (e.g., ~0.5 mm). When the application of the load is located on the most anterior aspect of the articular surface (Area C), then the impact may be clinically relevant (e.g., more than 1 mm).

A third limitation relates to the source of error associated with the placement of the device in the frontal (e.g., coronal) plane as it would impact the distribution of the moment arms and therefore the balance. Similar to the discussion above, in some cases, this may be significant for bi-compartmental joints.

A fourth limitation relates to the difficulty of maintaining a true force-controlled feedback loop of the expandable member. Therefore, depending on the height, the distraction force may fluctuate.

The exemplary embodiments relate to ligament balancing devices that address one or more of the shortcomings described above. In some embodiments described herein, exemplary ligament balancing devices will be described with reference to the total knee joint. In such devices, a first plate (e.g., a proximal plate) is configured to contact the distal aspect of a patient’s femur (e.g., the patient’s native femur), a trial femoral component, or a femoral component depending on the stage of the surgery (e.g., whether performed prior to or subsequent to femoral cuts); and a second plate (e.g., a distal plate is configured to contact a proximal end of the patient’s tibia (e.g., a cut surface of a proximal end of the tibia or the proximal end of the native tibia). In other embodiments, the concepts embodied by the exemplary embodiments of this disclosure can be applied to any joint. For example, in some embodiments, the bicondylar ligament balancing devices of the exemplary embodiments can be divided into two at the level of the sagittal plane of symmetry and each side specific sub-component can be used for partial knee joint or total knee joint where the cruciate ligaments are maintained in place. Similarly, in some embodiments, the exemplary ligament balancing devices described herein may be adapted for use in other joints, such as a shoulder joint (in which case a first plate may be a medial plate configured to contact an aspect of the glenoid and a second plate may be a lateral plate configured to contact an aspect of the humerus, or vice versa), an ankle joint (in which case a first plate may be a proximal plate configured to contact an aspect of the distal end of the tibia and a second plate may be a distal plate configured to contact an aspect of the talus, or vice versa), a hip joint, an elbow joint, etc. The exemplary embodiments described herein use the term “plate” to refer to various elements that are adapted to act as points of contact between the exemplary ligament balancing devices described herein and the bony surfaces of a joint. The specific shapes of plates described herein are only exemplary and other differently shaped contact elements are possible without departing from the broader concepts disclosed herein. For example, a plate need not include a contiguous and/or uninterrupted contact surface, and may include one or more holes or other interruptions therein. Additionally, a plate need not be planar, though it may be, and may also be at least partially concave or at least partially convex.

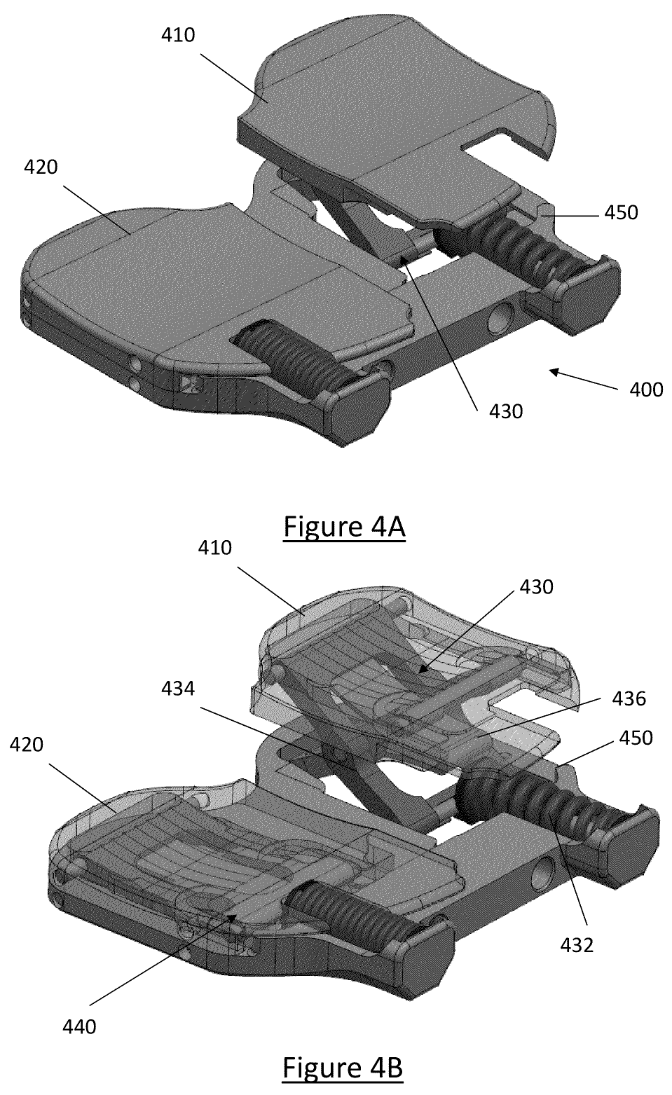

FIGS. 4A and 4B show perspective views of a first exemplary ligament balancing device 400, with the perspective view of FIG. 4B showing certain elements of the ligament balancing device 400 in a translucent manner so as to allow inner components of the ligament balancing device 400 to be seen. In some embodiments, an exemplary ligament balancing device 400 includes two separate first plates 410 and 420, wherein the first one of the first plate 410 is intended to engage with a first condyle of the femur (e.g., either one of the medial condyle or the lateral condyle) and the second one of the first plate 420 is intended to engage with the second condyle of the femur (e.g., the other one of either the medial condyle or the lateral condyle). In some embodiments, the exemplary ligament balancing device 400 includes a second plate 450 that is configured to engage a tibia. In some embodiments, a first expansion mechanism 430 is located between the first one of the first plate 410 and the second plate 450 and a second expansion mechanism 440 is located between the second one of the first plate 420 and the second plate 450. In some embodiments, the first expansion mechanism 430 and the second expansion mechanism 440 are independent from one another. In such embodiments, the range of angular tilt is not linked to (and therefore not limited by) the value of the height. For example, as shown in FIGS. 4A and 4B, if the first one of the first plate 410 is in a fully collapsed position with respect to the second plate 450, then the second one of the first plate 420 can still independently self-adjust through its entire range of height depending on the ligament laxity.

In some embodiments, the first expansion mechanism 430 includes a compression spring 432 (e.g., which is or forms part of a first actuation sub-mechanism), an expanding scissor mechanism 434, and a scissor leaf spring 436 (e.g., which is or forms part of a second actuation sub-mechanism). In some embodiments, the compression spring 432 is positioned so as to apply a force between the second plate 450 and the expanding scissor mechanism 434. In some embodiments, the scissor leaf spring 436 is positioned within the expanding scissor mechanism 434 so as to apply to a force to the expanding scissor mechanism. In some embodiments, both the compression spring 432 and the scissor leaf spring 436 are configured so as to apply corresponding forces that urge the first one of the first plate 410 away from the second plate 450. For clarity, only the elements of the first expansion mechanism 430 are labeled in FIGS. 4A and 4B; in some embodiments, the second expansion mechanism 440 includes the same elements as does the first expansion mechanism 430.

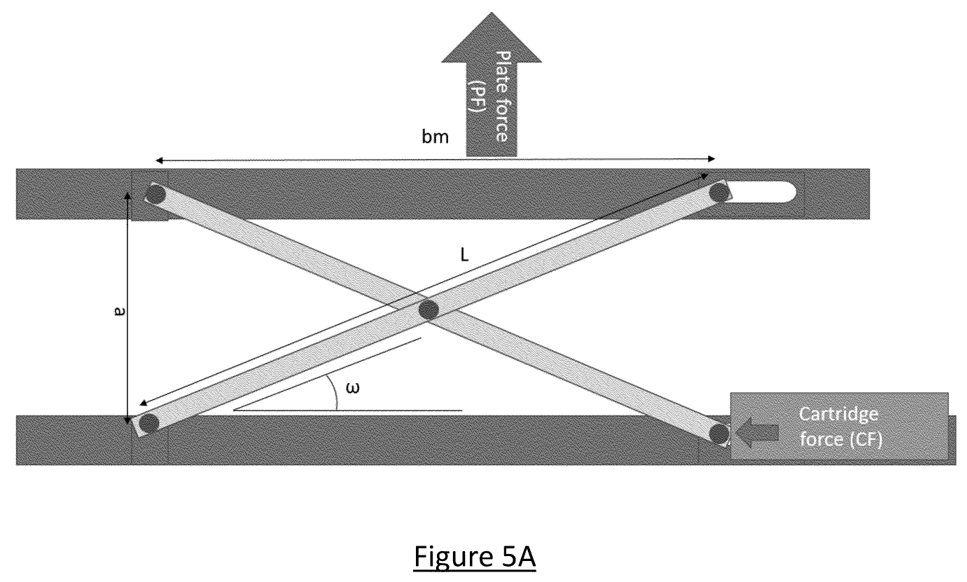

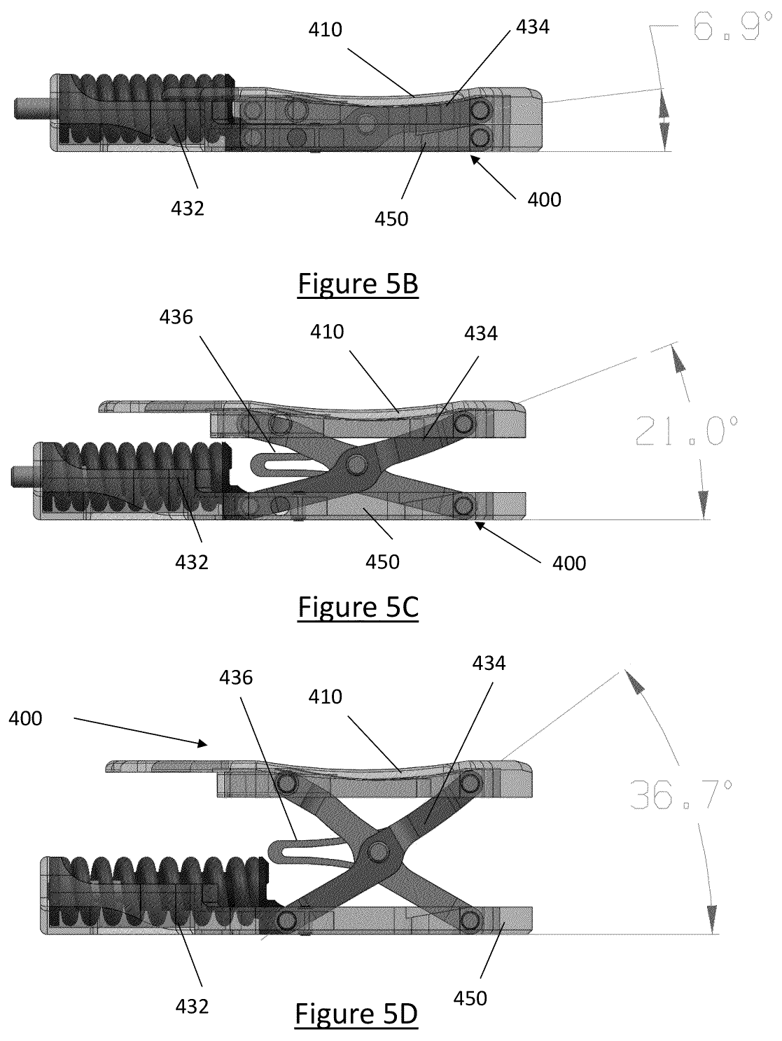

FIGS. 5B, 5C, and 5D show side views of the ligament balancing device 400 in which the first one of the first plate 410 is positioned in compressed, intermediate, and expanded positions, respectively. In some embodiments, the first expansion mechanism 430 is configured such that the distraction force applied by the first expansion mechanism 430 is a substantially constant distraction force across the range of expansion of the first plate 410 relative to the second plate 450.

As used herein, the terms “substantially constant distraction force” and “quasi-constant force,” as used to describe the distraction force applied by an exemplary ligament balancing device across an available range of expansion of such a ligament balancing device (e.g., from a most compressed position to a most expanded position of the first plate 410 relative to the second plate 450), refers to a force that varies by no more than a certain variance percentage as compared to a nominal distraction force (i.e., is no more than the certain percentage greater than or less than the nominal distraction force). For example, if the nominal distraction force of an exemplary ligament balancing device is ten (10) pounds and the certain percentage is 10%, then a “substantially constant distraction force” is a force that is within plus or minus 10% of the nominal value of ten (10) pounds, i.e., is between nine (9) and eleven (11) pounds. In some embodiments, the variance percentage is 5%. In some embodiments, the variance percentage is less than or equal to 5%. In some embodiments, the variance percentage is 10%. In some embodiments, the variance percentage is less than or equal to 10%. In some embodiments, the variance percentage is 11%. In some embodiments, the variance percentage is less than or equal to 11%. In some embodiments, the variance percentage is 12%. In some embodiments, the variance percentage is less than or equal to 12%. In some embodiments, the variance percentage is 13%. In some embodiments, the variance percentage is less than or equal to 13%. In some embodiments, the variance percentage is 14%. In some embodiments, the variance percentage is less than or equal to 14%. In some embodiments, the variance percentage is 15%. In some embodiments, the variance percentage is less than or equal to 15%. In some embodiments, the variance percentage is 16%. In some embodiments, the variance percentage is less than or equal to 16%. In some embodiments, the variance percentage is 17%. In some embodiments, the variance percentage is less than or equal to 17%. In some embodiments, the variance percentage is 18%. In some embodiments, the variance percentage is less than or equal to 18%. In some embodiments, the variance percentage is 19%. In some embodiments, the variance percentage is less than or equal to 19%. In some embodiments, the variance percentage is 20%. In some embodiments, the variance percentage is less than or equal to 20%.

Continuing to refer to FIGS. 5B, 5C, and 5D, in some embodiments, the force applied by the compression spring 432 to the expanding scissor mechanism 434 decreases as the first expansion mechanism 430 expands (e.g., as the distance between the first one of the first plate 410 and the second plate 450 increases). In some embodiments, the moment arm of the force applied by the compression spring 432 (e.g., a physical parameter of the first expansion mechanism 430) about the fulcrum of the expanding scissor mechanism 434 increases as the first expansion mechanism 430 expands. Consequently, in some embodiments, the distraction force applied between the first one of the first plate 410 and the second plate 450 due to the force applied by compression spring 432 increases as the first expansion mechanism 430 expands as a result of the increasing moment arm. In some embodiments, the distraction force applied to the expanding scissor mechanism 434 by the scissor leaf spring 436 decreases as the first expansion mechanism 430 expands. As a result, the distraction forces applied by the compression spring 432 (as modified by the change in the moment arm of the expanding scissor mechanism 434) and the scissor leaf spring 436 can be described as antagonist to one another. In some embodiments, the distraction forces applied by the compression spring 432 (as modified by the change in the moment arm of the expanding scissor mechanism 434) and the scissor leaf spring 436 combine to provide the substantially constant distraction force between the first one of the first plate 410 and the second plate 450. This combination is illustrated by the following description with reference to each of FIGS. 5A, 5B, 5C, and 5D. In the description below, the nominal distraction force to be applied by the first expansion mechanism 430 is 110 Newtons. In other embodiments, similar results can be obtained for different substantially constant distraction forces.

A scissor mechanism (e.g., the expanding scissor mechanism 434) allows two plates (e.g., the first one of the first plate 410 and the second plate 450) to be maintained parallel to each other and to move with respect to one another in an axial direction (e.g., so as to provide a distraction force to two opposing bony structures of a joint as described herein). Relevant dimensions for characterizing such a scissor mechanism are described below with reference to FIG. 5A, which shows a scissor mechanism at maximum expansion. In a scissor mechanism, opposing plates are separated by a given distance “a”, the scissor horizonal length at a maximum value of height “a” can be referred to as “bm”, the angle of the scissor mechanism relative to the plates can be defined as “ω”, a length of one leg of the scissor legs is referred to as “L”. In a scissor mechanism configured in a manner such as the expanding scissor mechanism 434 described above, a compression force “CF” (e.g., as applied by the compression spring 432) is applied at the end of one of the scissor legs, resulting in the generation of a plate force “PF” (e.g., a distraction force). In cases where the compression force CF is applied by a compressing spring (e.g., the compression spring 432), the spring can be characterized as having a free length “FL” (e.g., the length when no force is applied), a displacement “D” from the free length, and a Hookean spring constant “k” defining the force output of the spring per unit of compression. In such cases, the plate force PF can be calculated as:

$$P F = C F \times tan \text{ω}$$

In the expression above, assuming that there is no friction, the compression force CF can be calculated as:

$$C F = \left(F L - D\right) k$$

And the displacement D can be calculated as:

$$D = \sqrt{{L}^{2} - {a}^{2}} - \text{bm}$$

Referring now to FIG. 5B, the ligament balancing device 400 is shown with the first one of the first plate 410 in a fully compressed position, in which the space between the first one of the first plate 410 and the second plate 450 is 6 millimeters (as measured between the contact surfaces of the respective plates, e.g., from the proximal aspect of the first plate 410 to the distal aspect of the second plate 450). In the position shown in FIG. 5B, the compression spring 432 applies a force of 257 Newtons to the expanding scissor mechanism 434. In the position shown in FIG. 5B, the angle of the scissor fulcrum is 6.9 degrees. As a result, in the position shown in FIG. 5B, the force applied by the compression spring 432 to the first one of the first plate 410, as influenced by the moment arm of the expanding scissor mechanism 434, is 257 Newtons × tan 13.7 degrees = 31 Newtons (rounded to the nearest even number). In the position shown in FIG. 5B, the distraction force applied by the scissor leaf spring 436 to the expanding scissor mechanism 434, and thereby directly to the first one of the first plate 410, is 83 Newtons. As such, the total distraction force applied between the first one of the first plate 410 and the second plate 450, when positioned as shown in FIG. 5B, is 114 Newtons.

Referring now to FIG. 5C, the ligament balancing device 400 is shown with the first one of the first plate 410 in a partially expanded position, in which the space between the first one of the first plate 410 and the second plate 450 is 12 millimeters (as measured between the contact surfaces of the respective plates). In the position shown in FIG. 5C, the compression spring 432 applies a force of 186 Newtons to the expanding scissor mechanism 434. In the position shown in FIG. 5C, the angle of the scissor fulcrum is 21.0 degrees. As a result, in the position shown in FIG. 5C, the force applied by the compression spring 432 to the first one of the first plate 410, as influenced by the moment arm of the expanding scissor mechanism 434, is 75 Newtons. In the position shown in FIG. 5C, the distraction force applied by the scissor leaf spring 436 to the expanding scissor mechanism 434, and thereby directly to the first one of the first plate 410, is 50 Newtons. As such, the total distraction force applied between the first one of the first plate 410 and the second plate 450, when positioned as shown in FIG. 5C, is 124 Newtons.

Referring now to FIG. 5D, the ligament balancing device 400 is shown with the first one of the first plate 410 in a further expanded position, in which the space between the first one of the first plate 410 and the second plate 450 is 18 millimeters (as measured between the contact surfaces of the respective plates). In the position shown in FIG. 5D, the compression spring 432 applies a force of 116 Newtons to the expanding scissor mechanism 434. In the position shown in FIG. 5D, the angle of the scissor fulcrum is 36.7 degrees. As a result, in the position shown in FIG. 5D, the force applied by the compression spring 432 to the first one of the first plate 410, as influenced by the moment arm of the expanding scissor mechanism 434, is 86 Newtons. In the position shown in FIG. 5D, the distraction force applied by the scissor leaf spring 436 to the expanding scissor mechanism 434, and thereby directly to the first one of the first plate 410, is 15 Newtons. As such, the total distraction force applied between the first one of the first plate 410 and the second plate 450, when positioned as shown in FIG. 5D, is 101 Newtons.

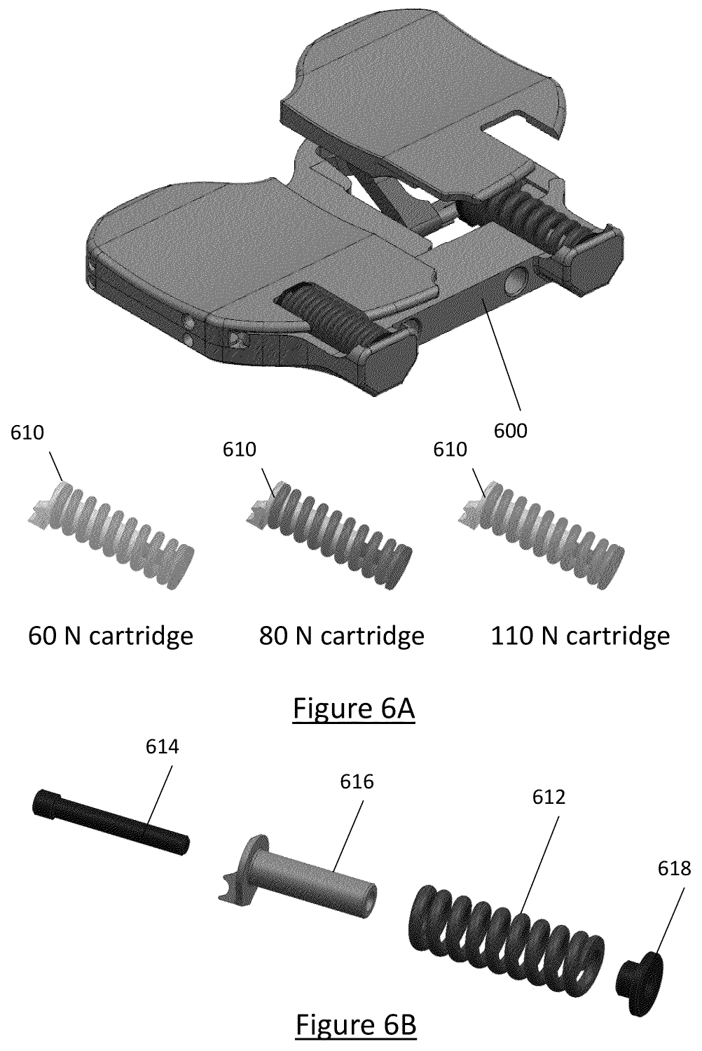

In some embodiments, the exemplary ligament balancing device 400 is formed as an “exoskeleton” that is configured to allow different versions of the compression spring 432 to be positioned interchangeably within the exemplary ligament balancing device 400. In some embodiments, different ones of the compression spring 432 are configured as cartridges to facilitate such interchangeability. FIG. 6A illustrates the exemplary ligament balancing device exoskeleton 600 along with three such cartridges 610. For example, in some embodiments, cartridges 610 are configured so as to provide a substantially constant distraction force of 60 Newtons, 80 Newtons, and 110 Newtons. FIG. 6B shows an exploded view of an exemplary cartridge 610. In some embodiments, each cartridge 610 includes a compression spring 612, a piston 614, a cylinder 616, and a piston cap 618. In some embodiments, the piston 614 is joined with the piston cap 618 and moves within the cylinder 616 so as to guide the compression spring 612 to compress and expand only in an axial direction. In some embodiments, the exemplary ligament balancing device exoskeleton 600 and various ones of the cartridge 610 are provided as elements of a kit so as to allow a user (e.g., a surgeon) to independently select the substantially constant distraction force to be applied to the first one of the first plate 410 and to the second one of the first plate 420 relative to the second plate 450.

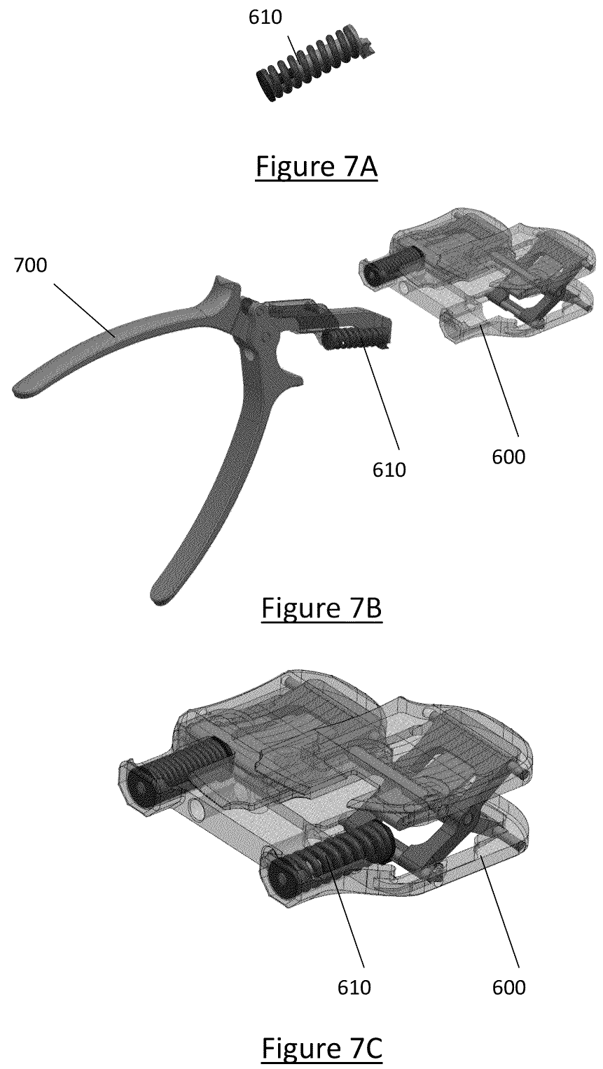

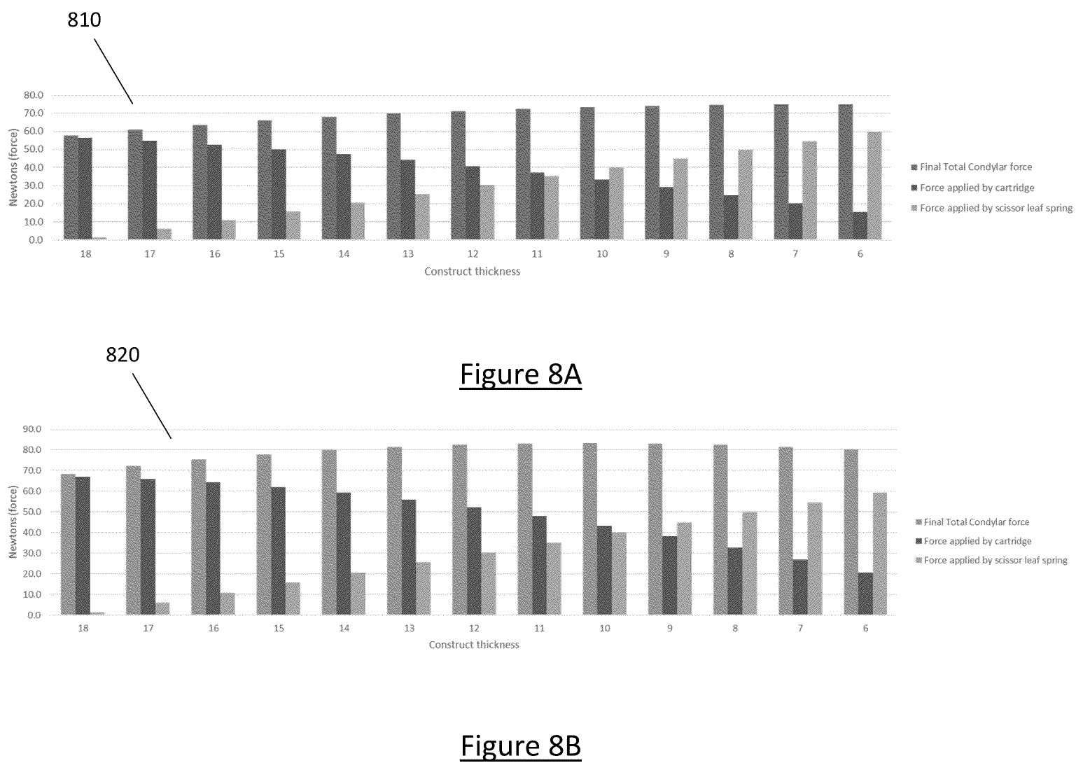

In some embodiments, such selection and adjustment can be performed as a “back table” adjustment. In some embodiments, an exemplary kit also includes an insertion device operable to insert a selected one of the cartridges 610 into the ligament balancing device exoskeleton 600. FIGS. 7A, 7B, and 7C show the steps of assembly of the exoskeleton 600 and cartridges 610. First, a user of the kit (e.g., a surgeon) selects a desired one of the cartridges 610 based on the desired distraction force to be applied either to the first one of the first plate 410 or the second one of the first plate 420. FIG. 7A shows a selected one of the cartridges 610 (e.g., a cartridge 610 configured to provide a distraction force of 80 Newtons). Second, a user of the kit (e.g., a surgeon) loads the selected one of the cartridges 610 into an insertion device 700. FIG. 7B shows an exemplary insertion device 700 holding the selected one of the cartridges 610 and prepared for insertion into the ligament balancing device exoskeleton 600. In some embodiments, the insertion device 700 is configured to retain the selected one of the cartridges 610 in a fully compressed position so as to facilitate insertion into the ligament balancing device exoskeleton 600. Third, a user of the kit (e.g., a surgeon) places the selected one of the cartridges 610 into the ligament balancing device exoskeleton 600 using the insertion device 700. FIG. 7C shows the ligament balancing device exoskeleton 600 having the selected one of the cartridges 610 inserted therein.

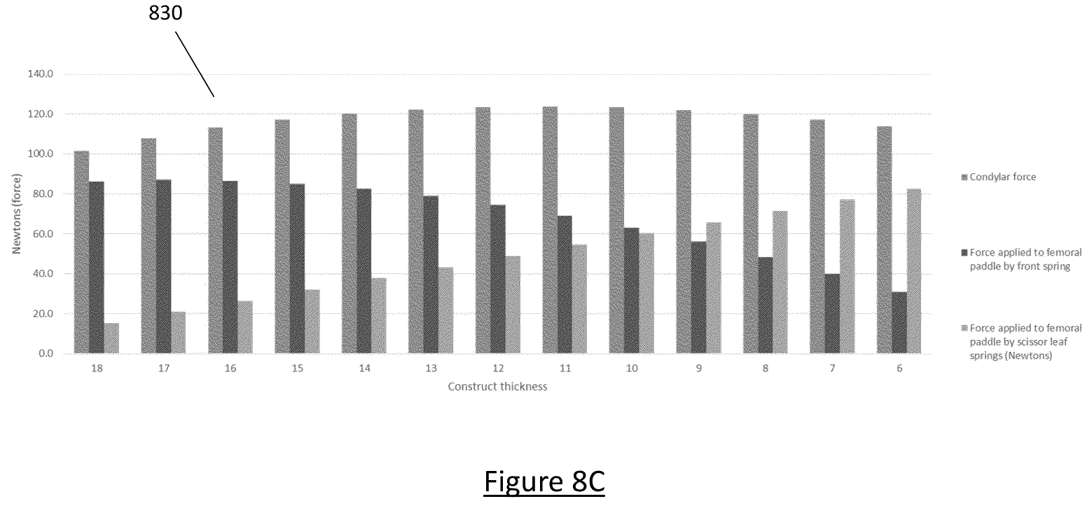

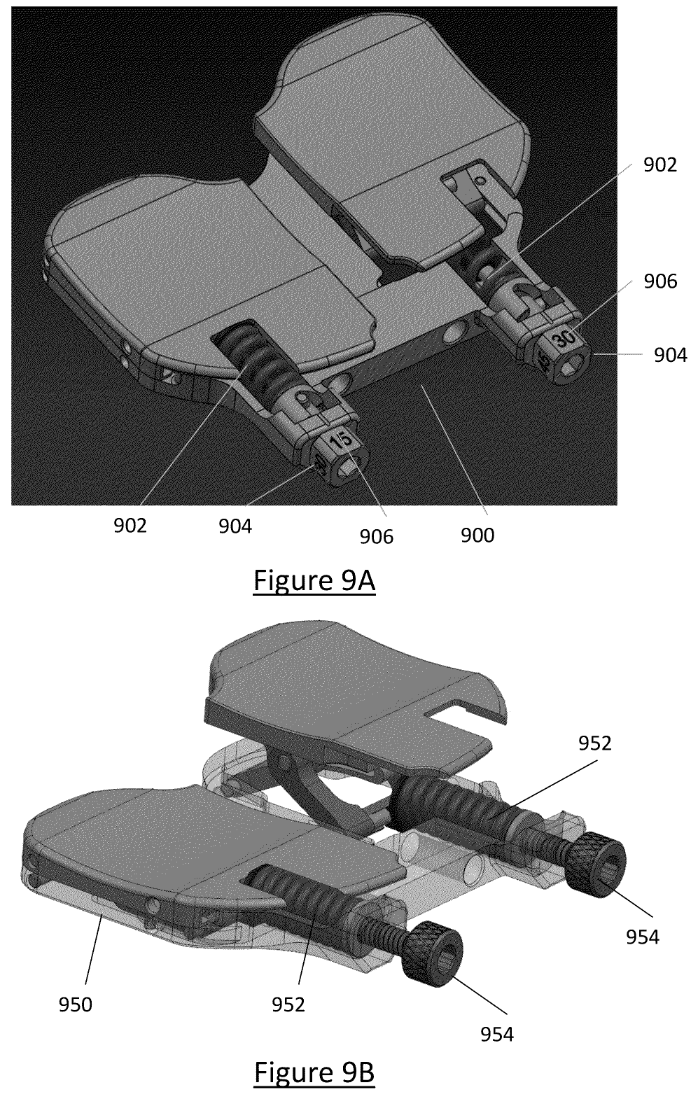

As described above, cartridges 610 can be provided to allow the ligament balancing device 400 assembled from the ligament balancing device exoskeleton 600 and selected ones of the cartridges 610 to provide desired substantially constant distraction forces to be applied to both the first one of the first plate 410 and the second one of the first plate 420. FIGS. 8A, 8B, and 8C show graphs 810, 820, and 830, respectively, of the forces applied at various points along the range of expansion by the ligament balancing device 400 including a cartridge 610 designed to provide a 60 Newton distraction force, a cartridge 610 designed to provide an 80 Newton distraction force, and a cartridge 610 designed to provide a 110 Newton distraction force, respectively. Each data point in each of the graphs 810, 820, and 830 shows, for a given point in the range of expansion of the ligament balancing device 400, the total force applied to the corresponding one of the first plates 410 or 420 (leftmost bar), the portion of the total force applied by the corresponding one of the cartridges 610 as influenced by the moment arm of the expanding scissor mechanism 434 at the given point in the range of expansion (central bar), and the portion of the total force applied by the scissor leaf spring 436 (rightmost bar). As may be seen from FIGS. 8A, 8B, and 8C, each of the cartridges 610 provides a substantially constant distraction force across the range of expansion. Although FIGS. 8A, 8B, and 8C show corresponding graphs 810, 820, and 830 for cartridges 610 that are designed to provide distraction forces of 60 Newtons, 80 Newtons, and 110 Newtons, respectively, in other embodiments, cartridges 610 are configured to provide different magnitudes of substantially constant distraction force. In some embodiments, the cartridges 610 provide substantially constant distraction forces that are in a range of from 10 Newtons to 300 Newtons. In some embodiments, the cartridges 610 provide substantially constant distraction forces that are in a range of from 50 Newtons to 150 Newtons.

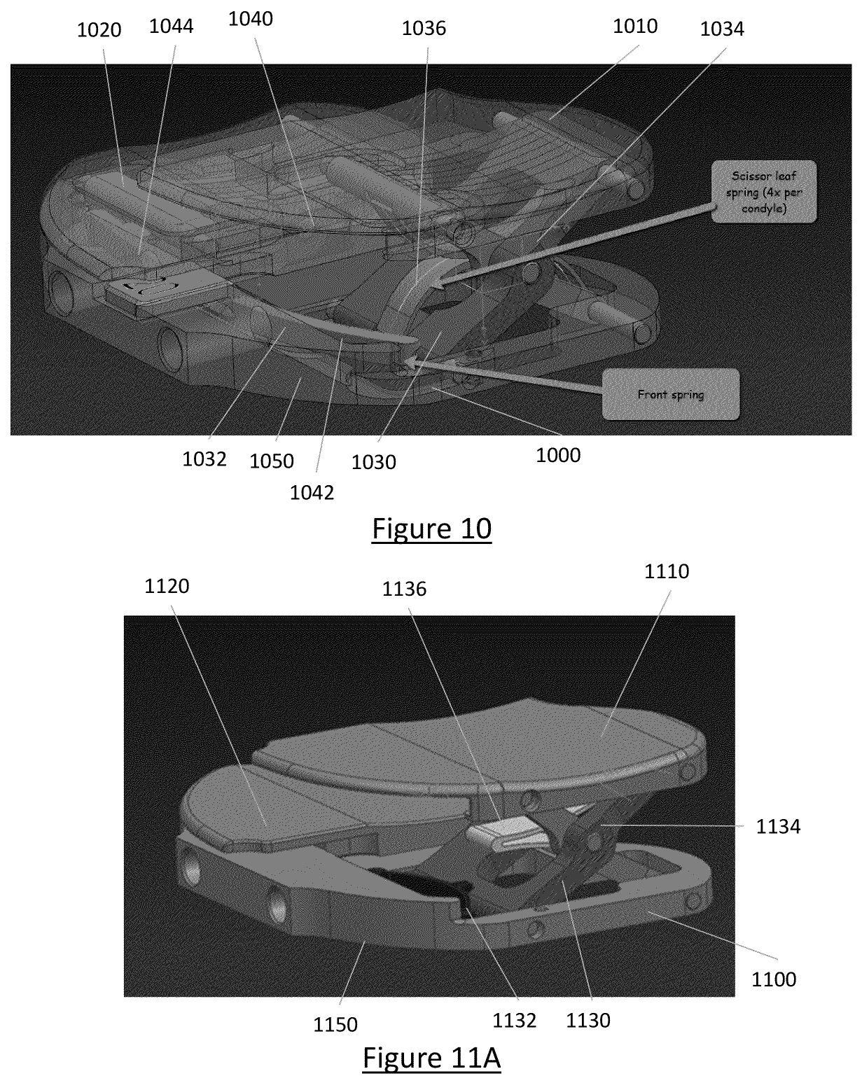

In some embodiments, a ligament balancing device is configured to allow a user (e.g., a surgeon) to adjust the substantially constant distraction force applied to each condyle without interchangeable components. FIGS. 9A and 9B, illustrate some such embodiments. FIG. 9A shows an exemplary embodiment of a ligament balancing device 900 that includes compression springs 902 that are adjustable. In some embodiments, the compression springs 902, in conjunction with other elements of the ligament balancing device 900, are operable to provide a substantially constant distraction force across the range of expansion of the ligament balancing device 900. In some embodiments, the compression springs 902 can be adjusted by positioning corresponding screws 904. In some embodiments, different positions of the screw 904 result in different preloads applied to the compression springs 902, thereby adjusting the substantially constant distraction force. In some embodiments, the screws 904 are configured to provide discretely defined fixed values for the substantially constant distraction force from the corresponding compression springs 902. For example, in some embodiments, the screws 904 include stops or other mechanical elements that allow the screws 904 to be positioned in a plurality of discrete positions. In some embodiments, the screws 904 include markings 906 that describe the different substantially constant distraction forces provided by the ligament balancing device 900 at different adjustment levels of the screws 904. In some embodiments, the ligament balancing device 900 can be adjusted in situ or as a “back table” adjustment.

FIG. 9B shows an exemplary embodiment of a ligament balancing device 950 that includes compression springs 952 that are adjustable. In some embodiments, the compression springs 952, in conjunction with other elements of the ligament balancing device 950, are operable to provide a substantially constant distraction force across the range of expansion of the ligament balancing device 950. In some embodiments, the compression springs 952 can be adjusted by positioning corresponding screws 954. In some embodiments, different positions of the screw 954 result in different preloads applied to the compression springs 952, thereby adjusting the substantially constant distraction force. In some embodiments, the screws 954 are configured to provide a continuously adjustable range of distraction force. In some embodiments, the ligament balancing device 950 is configured to have a defined relationship between a torque applied to the screws 954 and the substantially constant distraction force provided by the ligament balancing device 950. In some embodiments, a user (e.g., a surgeon) can adjust the screws 954 using a torque-defined screwdriver to thereby configure the ligament balancing device 950 to provide a desired substantially constant distraction force. In some embodiments, the ligament balancing device 950 can be adjusted in situ or as a “back table” adjustment.

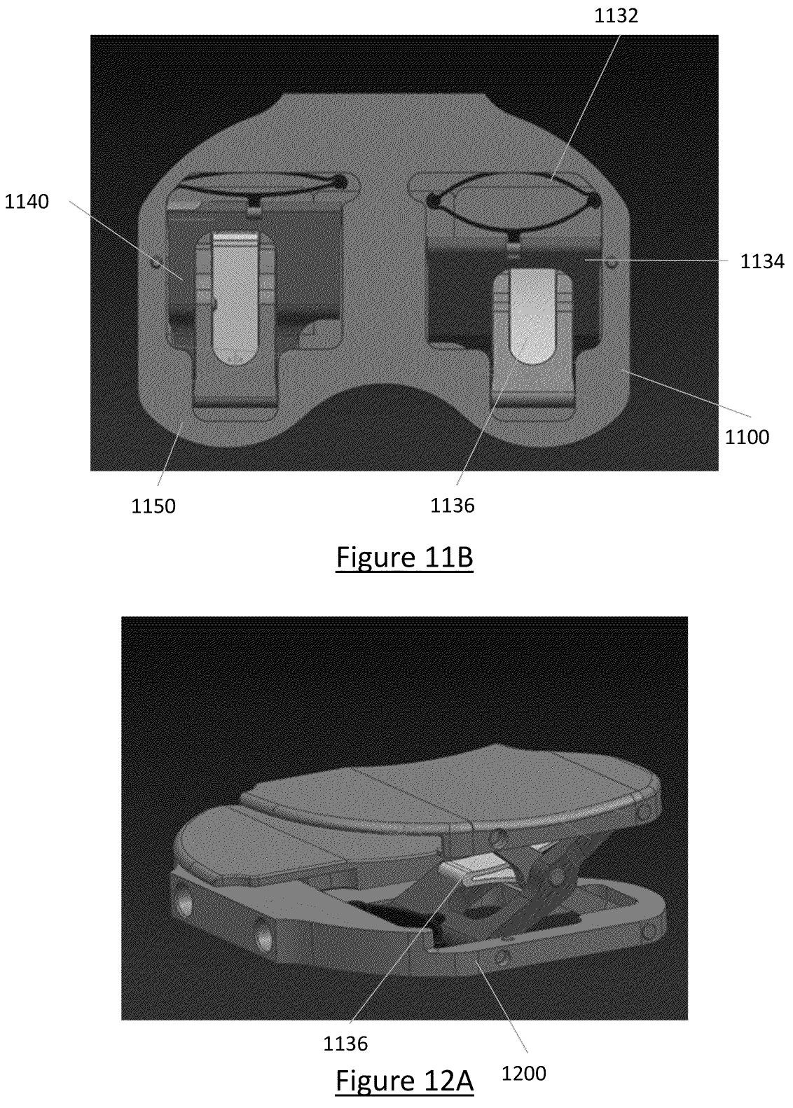

FIG. 10 shows an exemplary embodiment of a ligament balancing device 1000. In the embodiment shown in FIG. 10, the ligament balancing device 1000 includes two separate first plates 1010 and 1020, wherein the first one of the first plate 1010 is intended to engage with a first condyle of the femur (e.g., either one of the medial condyle or the lateral condyle) and the second one of the first plate 1020 is intended to engage with the second condyle of the femur (e.g., the other one of either the medial condyle or the lateral condyle). In some embodiments, the exemplary ligament balancing device 1000 includes a second plate 1050 that is configured to engage a tibia. In some embodiments, a first expansion mechanism 1030 is located between the first one of the first plate 1010 and the second plate 1050 and a second expansion mechanism 1040 is located between the second one of the first plate 1020 and the second plate 1050. In some embodiments, the first expansion mechanism 1030 and the second expansion mechanism 1040 are independent from one another. In such embodiments, the range of angular tilt is not linked to (and therefore not limited by) the value of the height. For example, as shown in FIG. 10, if the first one of the first plate 1010 is in a fully collapsed position with respect to the second plate 1050, then the second one of the first plate 1020 can still independently self-adjust through its entire range of height depending on the ligament laxity.

In some embodiments, the first expansion mechanism 1030 includes a leaf spring 1032, an expanding scissor mechanism 1034, and a scissor leaf spring 1036. In some embodiments, the scissor leaf spring 1036 includes four subsections, e.g., one subsection per “leg” of the scissor mechanism. In some embodiments, the leaf spring 1032 is positioned so as to apply a force between the second plate 1050 and the expanding scissor mechanism 1034. In some embodiments, the scissor leaf spring 1036 is positioned within the expanding scissor mechanism 1034 so as to apply to a force to the expanding scissor mechanism. In some embodiments, both the leaf spring 1032 and the scissor leaf spring 1036 are configured so as to apply corresponding forces that urge the first one of the first plate 1010 away from the second plate 1050. In some embodiments, the forces applied by the leaf spring 1032 (as modified by the varying moment arm of the expanding scissor mechanism 1034) and by the scissor leaf spring 1036 are antagonist to one another, thereby to cause the first expansion mechanism 1030 to provide a substantially constant distraction force across the range of expansion of the first expansion mechanism 1030. In some embodiments, the leaf spring 1032 includes a first side 1042 that forms a portion of the first expansion mechanism 1030 and a second side 1044 that forms a portion of the second expansion mechanism 1040. In some embodiments, the first side 1042 and the second side 1044 are configured so as to cause the first expansion mechanism 1030 and the second expansion mechanism 1040 to provide substantially constant distraction forces that are the same as one another. In some embodiments, the first side 1042 and the second side 1044 are configured so as to cause the first expansion mechanism 1030 and the second expansion mechanism 1040 to provide substantially constant distraction forces that differ from one another. In some embodiments, the ligament balancing device 1000 and the leaf spring 1032 are configured such that different ones of the leaf spring 1032 can be selectively positioned within the ligament balancing device 1000 so as to allow a user (e.g., a surgeon) to adjust the substantially constant distraction forces provided by the ligament balancing device 1000.

FIGS. 11A and 11B show an exemplary embodiment of a ligament balancing device 1100. FIG. 11A shows a perspective view and FIG. 11B shows a bottom view exposing interior elements of the ligament balancing device 1100. In the embodiment shown in FIGS. 11A and 11B, the ligament balancing device 1100 includes two separate first plates 1110 and 1120, wherein the first one of the first plate 1110 is positioned to engage with a first condyle of the femur (e.g., either one of the medial condyle or the lateral condyle) and the second one of the first plate 1120 is positioned to engage with the second condyle of the femur (e.g., the other one of either the medial condyle or the lateral condyle). In some embodiments, the exemplary ligament balancing device 1100 includes a second plate 1150 that is configured to engage a tibia. In some embodiments, a first expansion mechanism 1130 is located between the first one of the first plate 1110 and the second plate 1150 and a second expansion mechanism 1140 is located between the second one of the first plate 1120 and the second plate 1150. In some embodiments, the first expansion mechanism 1130 and the second expansion mechanism 1140 are independent from one another. In such embodiments, the range of angular tilt is not linked to (and therefore not limited by) the value of the height. For example, as shown in FIG. 11A, if the first one of the first plate 1110 is in a fully collapsed position with respect to the second plate 1150, then the second one of the first plate 1120 can still independently self-adjust through its entire range of height depending on the ligament laxity.

In some embodiments, the first expansion mechanism 1130 includes a leaf spring 1132, an expanding scissor mechanism 1034, and a scissor leaf spring 1036. In some embodiments, the leaf spring 1132 is positioned so as to apply a force between the second plate 1150 and the expanding scissor mechanism 1134. In some embodiments, the scissor leaf spring 1136 is positioned within the expanding scissor mechanism 1134 so as to apply to a force to the expanding scissor mechanism 1134. In some embodiments, both the leaf spring 1132 and the scissor leaf spring 1136 are configured so as to apply corresponding forces that urge the first one of the first plate 1110 away from the second plate 1150. In some embodiments, the force applied by the leaf spring 1132 (as modified by the varying moment arm of the expanding scissor mechanism 1134) and by the scissor leaf spring 1136 are antagonist to one another, thereby combining to cause the first expansion mechanism 1130 to provide a substantially constant distraction force across the range of expansion of the first expansion mechanism 1130. For brevity, only the elements of the first distraction mechanism 1130 are described herein; the second expansion mechanism 1140 is substantially identical to the first expansion mechanism 1130. In some embodiments, the first expansion mechanism 1130 and the second expansion mechanism 1140 are configured to provide substantially constant distraction forces that are the same as one another. In some embodiments, the first expansion mechanism 1130 and the second expansion mechanism 1140 are configured to provide substantially constant distraction forces that are different from one another.

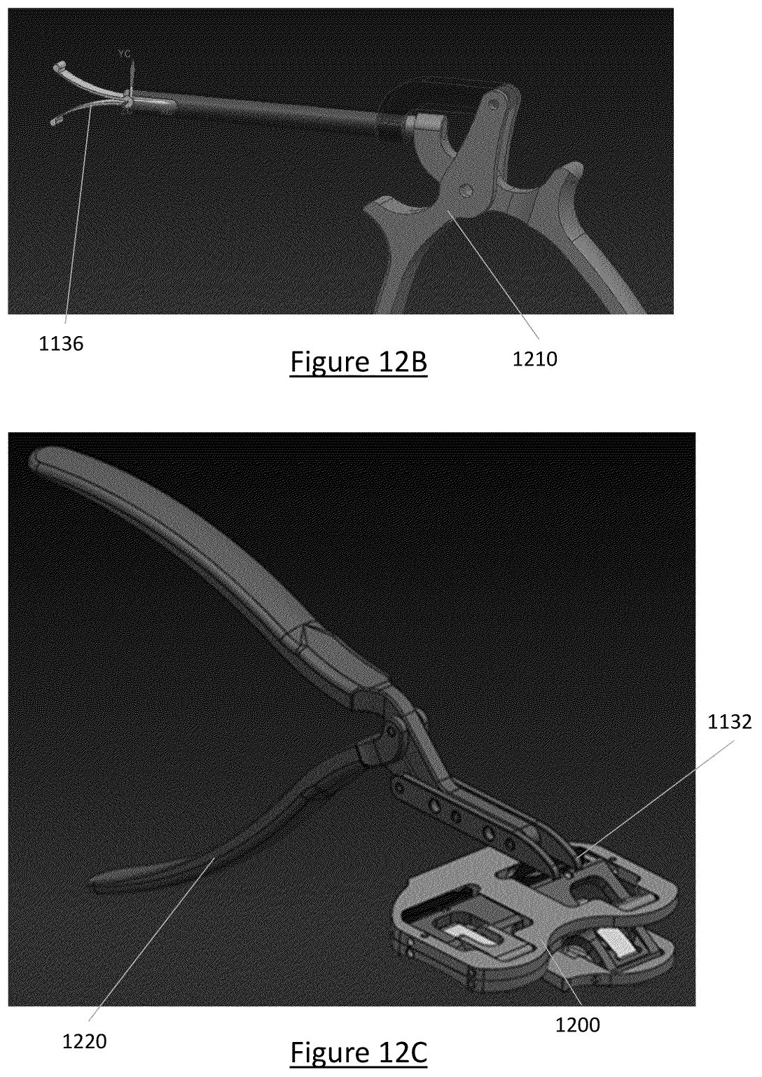

In some embodiments, the exemplary ligament balancing device 400 is formed as an “exoskeleton” that is configured to allow different versions of the leaf spring 1132 and/or the scissor leaf spring 1136 to be positioned interchangeably within an exoskeleton 1200 to form the exemplary ligament balancing device 1100. In some embodiments, different versions of the leaf spring 1132 and/or the scissor leaf spring 1136 are provided and are interchangeable in order to allow a user (e.g., a surgeon) to configure the substantially constant distraction force to be provided by the exemplary ligament balancing device 1100. FIGS. 12A, 12B, and 12C illustrate such an exemplary ligament balancing device exoskeleton 1200 and the method of use thereof. In some embodiments, selection and replacement of a desired leaf spring 1132 and/or a desired scissor leaf spring 1136 can be performed as a “back table” adjustment. In some embodiments, an exemplary kit also includes an insertion device 1210 operable to insert a selected one of the scissor leaf springs 1136 into the ligament balancing device exoskeleton 1200 and an insertion device 1220 operable to insert a selected one of the leaf spring 1132 into the ligament balancing device exoskeleton 1200. FIG. 12A shows an exemplary ligament balancing device exoskeleton 1200. FIG. 12B shows the exemplary insertion device 1210 engaging a selected scissor leaf spring 1136. FIG. 12C shows use of an insertion device 1220 to insert the selected one of the leaf springs 1132 into the ligament balancing device exoskeleton 1200.

The exemplary ligament balancing devices described above with reference to FIGS. 4A through 12B can be referred to as intraarticular ligament balancing devices, e.g., ligament balancing devices that are sized so as to be positioned inside a perimeter defined by the first plate, by the second plate, and/or by the opposing bony structures of the joint. In some embodiments, such a device can also be referred to as intracapsular. In some embodiments, an intraarticular device possesses advantages such as allowing for the possibility of closing the arthrotomy of a joint in order to better reflect a physiological kinematic of the joint during the manipulation of the joint, as well allowing for the reduction of the footprint of the device.

In other embodiments, an exemplary ligament balancing device is an extraarticularly-actuated ligament balancing device, e.g., a ligament balancing device having opposing plates that are positioned within the intraarticular space of a joint (e.g., between opposing bony structures of the joint) and other portions, including an actuation mechanism, that are positioned outside the intraarticular space. In some embodiments, an extraarticularly-actuated ligament balancing device includes opposing plates similar to the first and second plates described above that are configured to be positioned within the intraarticular space of a joint, and actuation mechanisms that are configured to remain outside the intraarticular space of the joint when such ligament balancing devices are in use. In such embodiments, the space between opposing plates, when in the most compressed position, can be made to be thinner than the space between opposing plates of an intraarticular device. For example, in some embodiments, the space between opposing plates of an extraarticularly-actuated device can be made as narrow as 1 millimeter. In contrast, due to the need to locate actuation elements between the plates, an intraarticular device is typically made no thinner than 6 millimeters in thickness in the most compressed position. As such, in some embodiments, an extraarticularly-actuated ligament balancing device is suited for characterizing the ligaments of a joint before any bone cuts are made, in which case the distance between opposing bones may be in the range of 1 millimeter to 5 millimeters, depending on the patient’s anatomy. Additionally, in some embodiments, an extraarticularly-actuated ligament balancing device is suited for characterizing the ligaments after only a first bone cut has been made, in which case the space between opposing bones may be as narrow as 3 millimeters depending on the patient’s anatomy, particularly in cases where a small bone cut is to be made such as in cases where a resurfacing implant is to be used.

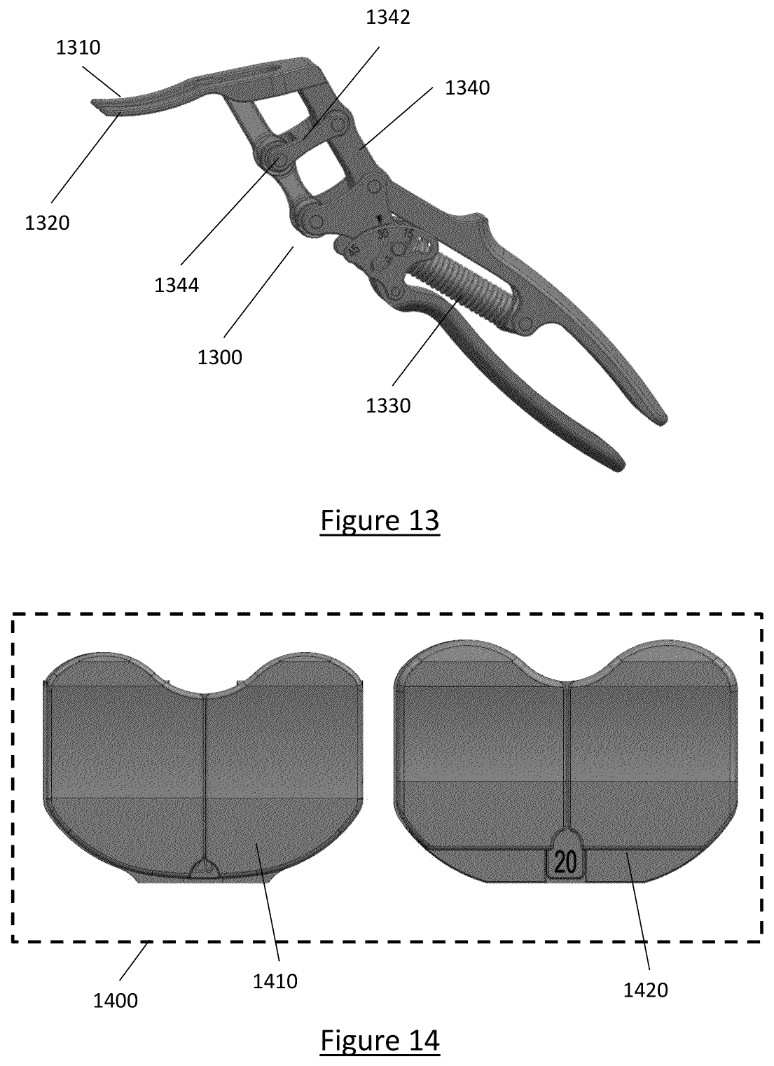

FIG. 13 shows an exemplary ligament balancing device 1300 that is an extraarticularly-actuated ligament balancing device. In some embodiments, the ligament balancing device 1300 includes at least one first plate 1310 and at least one second plate 1320.

In some embodiments, the at least one first plate 1310 includes one first plate 1310 that is configured (e.g., sized and shaped) to engage only one condyle of a first bone of a bicondylar joint (e.g., only the medial condyle of a femur of a knee joint or only the lateral condyle of a femur of a knee joint). In some embodiments, the at least one first plate 1310 includes one first plate 1310 that is configured (e.g., sized and shaped) to engage both condyles of a first bone of a bicondylar joint (e.g., both the medial condyle and the lateral condyle of a femur of a knee joint). In some embodiments, the at least one first plate 1310 includes two first plates 1310, each of which is configured (e.g., sized and shaped) to engage only one condyle of a first bone of a bicondylar joint (e.g., only the medial condyle of a femur of a knee joint or only the lateral condyle of a femur of a knee joint). In some embodiments, the at least one first plate 1310 includes one first plate 1310 that is configured (e.g., sized and shaped) to engage a condyle of a first bone of a unicondylar joint.

In some embodiments, the at least one second plate 1320 includes one second plate 1320 that is configured (e.g., sized and shaped) to engage only one condyle of a second bone (e.g., opposite the first bone) of a bicondylar joint (e.g., only the medial condyle of a tibia of a knee joint or only the lateral condyle of a tibia of a knee joint). In some embodiments, the at least one second plate 1320 includes one second plate 1320 that is configured (e.g., sized and shaped) to engage both condyles of a second bone of a bicondylar joint (e.g., both the medial condyle and the lateral condyle of a tibia of a knee joint). In some embodiments, the at least one second plate 1320 includes two second plates 1320, each of which is configured (e.g., sized and shaped) to engage only one condyle of a second bone of a bicondylar joint (e.g., only the medial condyle of a tibia of a knee joint or only the lateral condyle of a tibia of a knee joint). In some embodiments, the at least one second plate 1320 includes one second plate 1320 that is configured (e.g., sized and shaped) to engage a condyle of a second bone of a unicondylar joint.

In some embodiments, the ligament balancing device 1300 includes at least one mechanical force applying element 1330 (e.g., a compression spring). In some embodiments, the at least one mechanical force applying element 1330 is mechanically coupled to the at least one first plate 1310 and to the at least one second plate 1320 in a manner so as to urge the at least one first plate 1310 and the at least one second plate 1320 away from one another. In some embodiments, the ligament balancing device 1300 includes a mechanical linkage 1340 coupling the at least one mechanical force applying element 1330 to the at least one first plate 1310 and to the at least one second plate 1320. In some embodiments, the mechanical linkage 1340 includes at least one lever arm 1342 and at least one fulcrum 1344. In some embodiments, the mechanical linkage 1340 is configured such that an effective moment arm at which the at least one mechanical force applying element 1330 applies force to urge the at least one first plate 1310 away from the at least one second plate varies across the range of expansion between the at least one first plate 1310 and the at least one second plate 1320. In some embodiments, the force applied by the at least one mechanical force applying element 1330 varies across the range of expansion between the at least one first plate 1310 and the at least one second plate 1320. In some embodiments, the varying force applied by the at least one mechanical force applying element 1330 and the varying effective moment arm of the mechanical linkage 1340 combine to produce a substantially constant distraction force between the at least one first plate 1310 and the at least one second plate 1320 in a manner similar to that described above with reference to other exemplary ligament balancing devices. In some embodiments, the ligament balancing device 1300 is provided as part of a kit including multiple ones of the ligament balancing device 1300, each of which is configured to provide a different substantially constant distraction force.

In some embodiments, exemplary ligament balancing devices are provided in a kit including ligament balancing devices that are configured in a substantially similar manner to one another (e.g., all of the ligament balancing devices within a kit are configured to engage both condyles of a knee joint), but are sized differently from one another. FIG. 14 illustrates an exemplary kit 1400 that includes a small size ligament balancing device 1410 and a large size ligament balancing device 1420. The exemplary kit 1400 shown in FIG. 14 includes two ligament balancing devices, but in other embodiments any other number of ligament balancing devices may be included in a kit. In some embodiments, the ligament balancing devices included within a kit are configured to provide substantially constant distraction forces that are the same as one another. In some embodiments, the ligament balancing devices included within a kit are configured to provide substantially constant distraction forces that are different from one another. In some embodiments, the ligament balancing devices included within a kit are configured to provide substantially constant distraction forces that are adjustable, and which may realize adjustability in any of the manners described above.

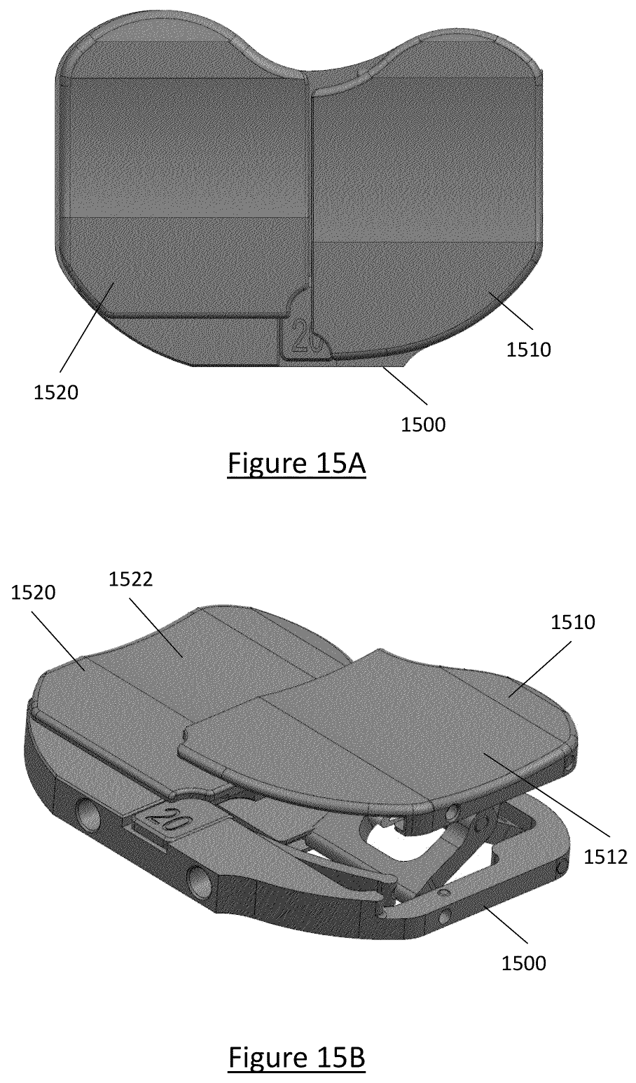

In some embodiments, any of the ligament balancing devices described herein may have first plates that are sized and shaped to suit the anatomy of the joint in which such ligament balancing devices tare to be used. For example, in some embodiments, an exemplary ligament balancing device that is configured for use in the knee joint includes two first plates that are sized and shaped to suit the anatomy of the medial compartment of the knee and the lateral compartment of the knee, respectively. FIGS. 15A and 15B show a top view and a perspective view, respectively, of an exemplary ligament balancing device 1500 having a medial first plate 1510 that is sized and shaped to suit the anatomy of the medial compartment of the knee joint, and a lateral first plate 1520 that is sized and shaped to suit the anatomy of the lateral compartment of the knee joint. In some embodiments, as shown in FIG. 15A, the medial first plate 1510 is longer in the anterior-posterior direction than is the lateral first plate 1520, corresponding to a typical patient’s anatomy in which the medial compartment of the knee is longer in the anterior-posterior direction than is the lateral compartment of the knee. In some embodiments, as shown in FIG. 15B, the medial first plate 1510 is narrower in the mediolateral direction than is the lateral first plate 1520, corresponding to a typical patient’s anatomy in which the medial compartment of the knee is narrower in the mediolateral direction than is the lateral compartment of the knee. In some embodiments, as shown in FIGS. 15A and 15B, the medial first plate 1510 and the lateral first plate 1520 have respective concave portions 1512 and 1522, corresponding to the convex shape of both the medial and lateral compartments of a native distal tibia. In some embodiments, as shown in FIGS. 15A and 15B, the concave portion 1522 of the lateral first plate 1520 has a greater degree of concavity than does the concave portion 1512 of the medial first plate, corresponding to the respective shapes of the medial and lateral compartments of a native distal tibia.

FIG. 16 shows a perspective view of an exemplary ligament balancing device 1600 having a medial first plate 1610 and a lateral first plate 1620. In the embodiment shown in FIG. 16, the medial first plate 1610 has a surface that is at least partially concave and the lateral first plate 1620 has a surface that is at least partially convex. In some embodiments, the ligament balancing device 1600 is suitable for use in knee applications in which the at least partially concave medial first plate 1610 emulates the at least partially concave medial plateau of a native tibia, and in which the at least partially convex lateral first plate 1620 emulates the at least partially convex.

The exemplary ligament balancing devices 1500 and 1600 described above configured for use in a knee joint following performance of a tibial cut and prior to performance of a femoral cut. In other embodiments, exemplary ligament balancing devices may include plates that are sized and shaped to interface with the bones of the knee joint at a different stage of a knee arthroplasty, or to interface with bones of another joint (e.g., a shoulder joint, an ankle joint, a hip joint, an elbow joint, etc.) at any stage of a respective arthroplasty.

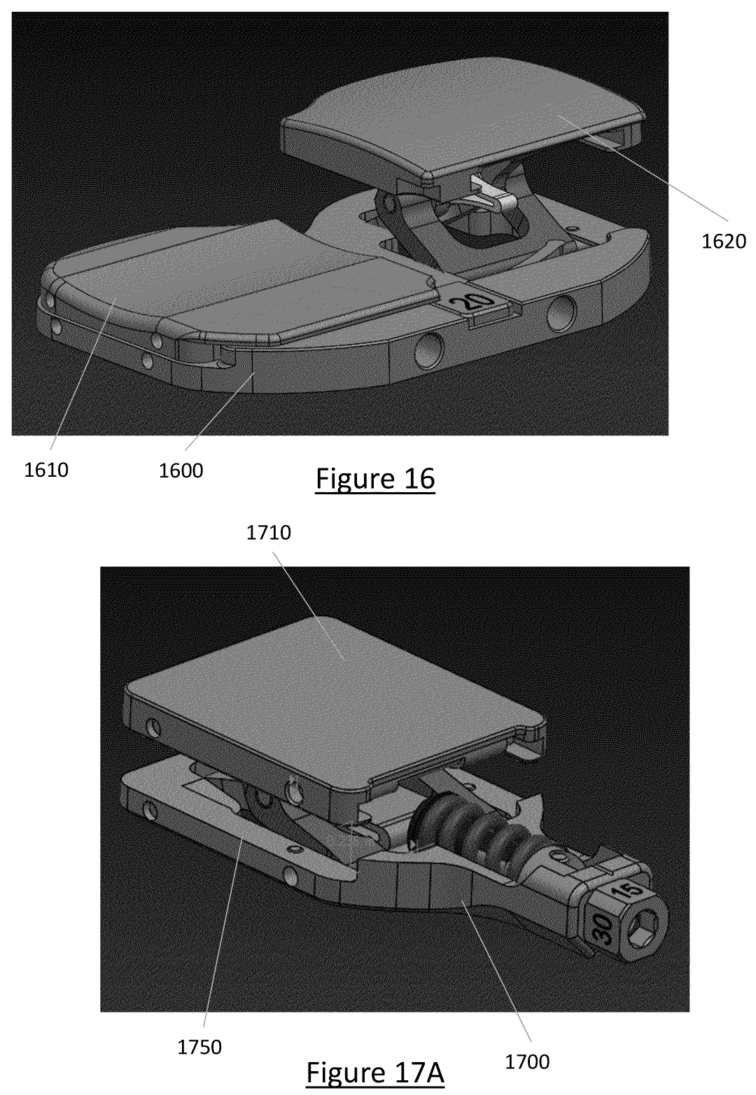

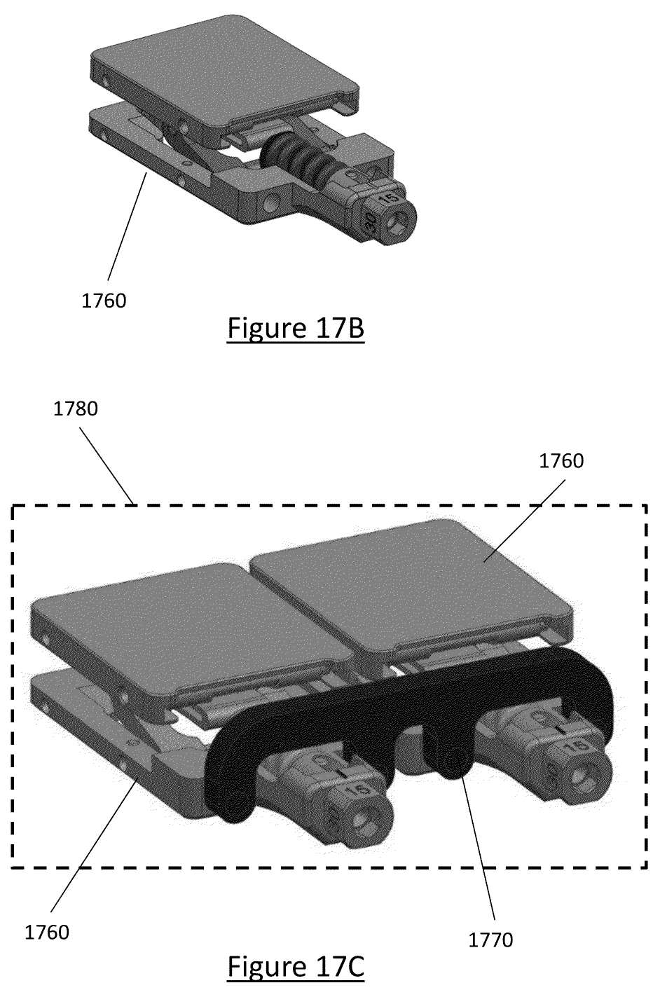

Various exemplary ligament balancing devices described herein, such as those described above with reference to FIGS. 4A and 9A, are configured for use during total arthroplasty of a bicondylar joint, such as during total knee arthroplasty, and therefore include two first plates that are configured to engage respective condyles of a bicondylar bone. In some embodiments, an exemplary ligament balancing device is configured for use during partial arthroplasty of a bicondylar joint, such as partial knee arthroplasty, and therefore includes one first plate that is configured to engage a single condyle of a bicondylar bone. FIG. 17A shows an exemplary ligament balancing device 1700 including a first plate 1710 and a second plate 1750. The ligament balancing device 1700 shown in FIG. 17A is configured for use during partial knee arthroplasty. In other embodiments, exemplary ligament balancing devices can also be configured for use during partial arthroplasty of other bicondylar joints. Additionally, the ligament balancing device 1700 shown in FIG. 17A includes a mechanism similar for adjusting a substantially constant distraction force similar to that of the ligament balancing device 900 shown in FIG. 9A. In other embodiments, exemplary unicondylar ligament balancing devices can be provided having a fixed or adjustable substantially constant distraction force that is supplied by any of the distraction mechanisms described herein.

In some embodiments, two unicondylar ligament balancing devices can be mechanically joined to form a bicondylar ligament balancing device. FIG. 17B shows an exemplary unicondylar ligament balancing device 1760 including a mechanism for attachment to another one of the unicondylar ligament balancing device 1760. FIG. 17C shows two of the exemplary ligament balancing device 1760 joined together by a mechanical linkage 1770 to form a bicondylar ligament balancing device 1780. the ligament balancing devices 1760 shown in FIGS. 17B and 17C includes a mechanism similar for adjusting a substantially constant distraction force similar to that of the ligament balancing device 900 shown in FIG. 9A. In other embodiments, exemplary unicondylar ligament balancing devices that are configured to allow for mechanical coupling to one another can be provided having a fixed or adjustable substantially constant distraction force that is supplied by any of the distraction mechanisms described herein.

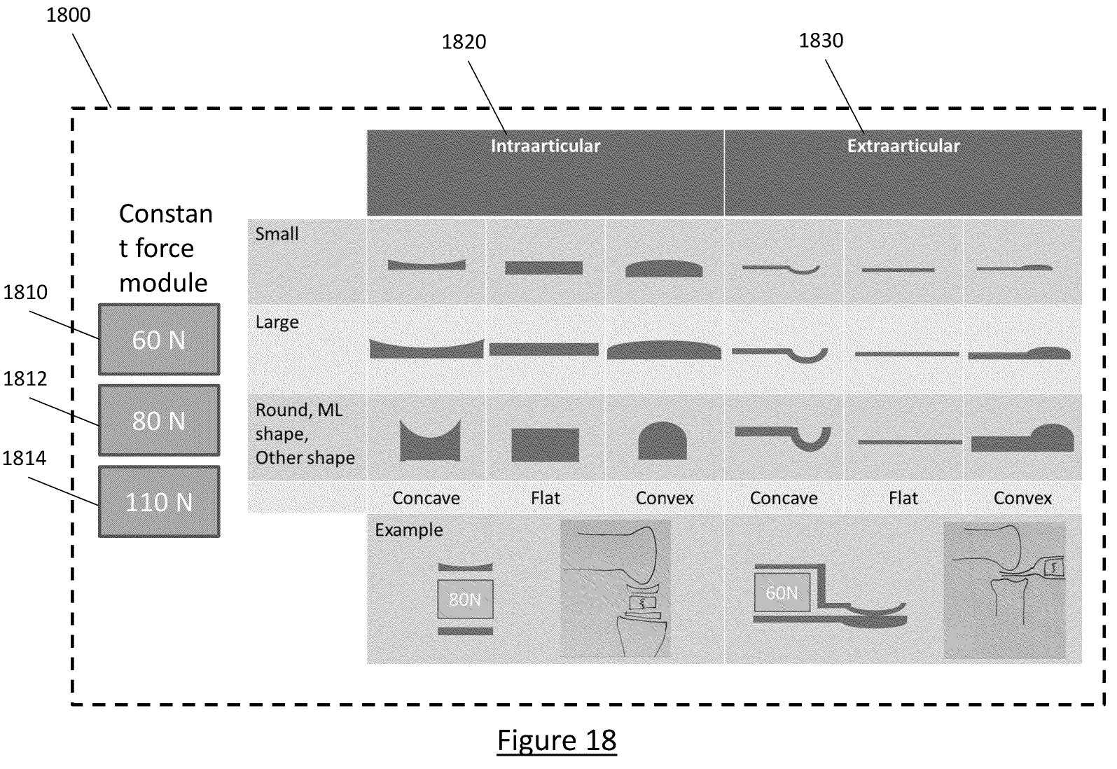

In some embodiments, an exemplary kit is provided that allows a user to select a distraction force and to select plates that are sized and shaped to apply force to opposing bones of varying shapes and at varying stages of a surgical procedure (e.g., before or after bone cuts have been performed). FIG. 18 shows an exemplary kit 1800. In some embodiments, the kit 1800 includes force modules 1810, 1812, 1814, each of which is configured to provide a different substantially constant distraction force. In some embodiments, the kit includes a set of intraarticular attachments 1820 configured to be coupled to a selected one of the force modules 1810, 1812, or 1814 to assemble a ligament balancing device configured for intraarticular use as described above. In some embodiments, the set of intraarticular attachments 1820 includes attachments configured to provide variously shaped contact surfaces (e.g., flat, concave, convex, etc.), each of which can be provided in a variety of sizes and shapes (e.g., a large size, a small size, a rounded shape, a medial-specific shape, a lateral-specific shape, etc.). In some embodiments, the kit includes a set of extraarticular attachments 1830 configured to be coupled to a selected one of the force modules 1810, 1812, or 1814 to assemble a ligament balancing device configured for intraarticular use as described above. In some embodiments, the set of extraarticular attachments 1830 includes attachments configured to provide variously shaped contact surfaces (e.g., flat, concave, convex, etc.), each of which can be provided in a variety of sizes and shapes (e.g., a large size, a small size, a rounded shape, a medial-specific shape, a lateral-specific shape, etc.).

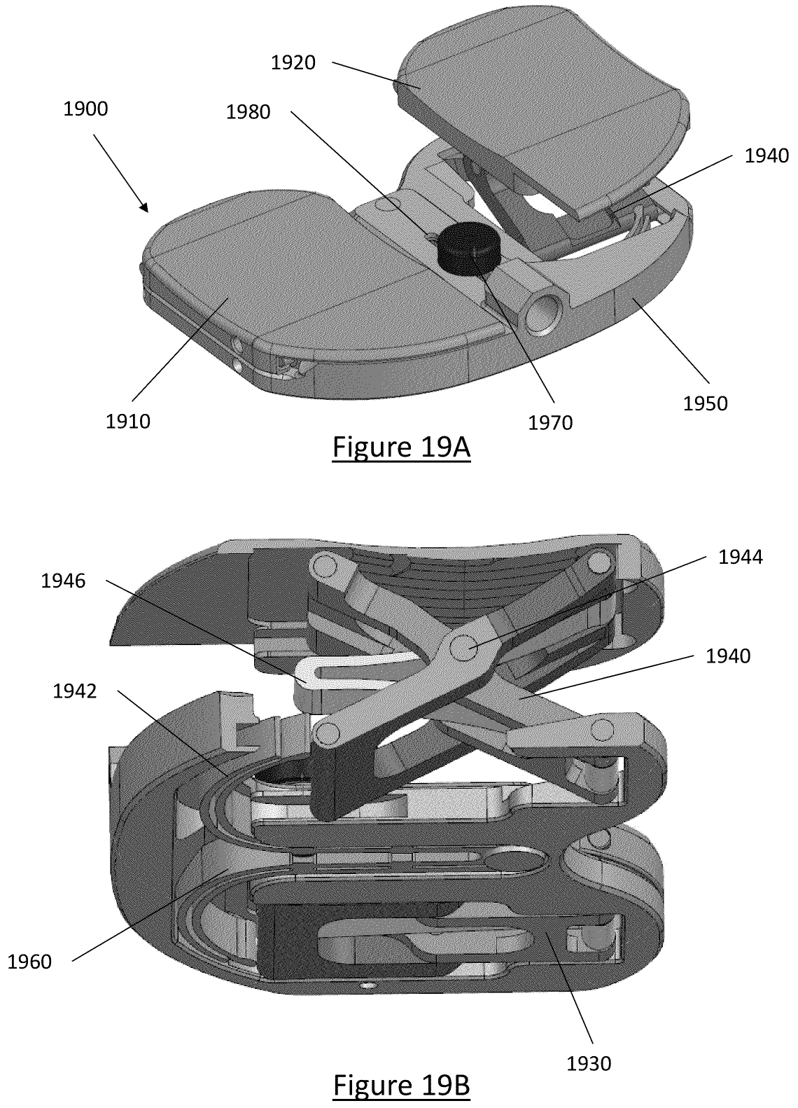

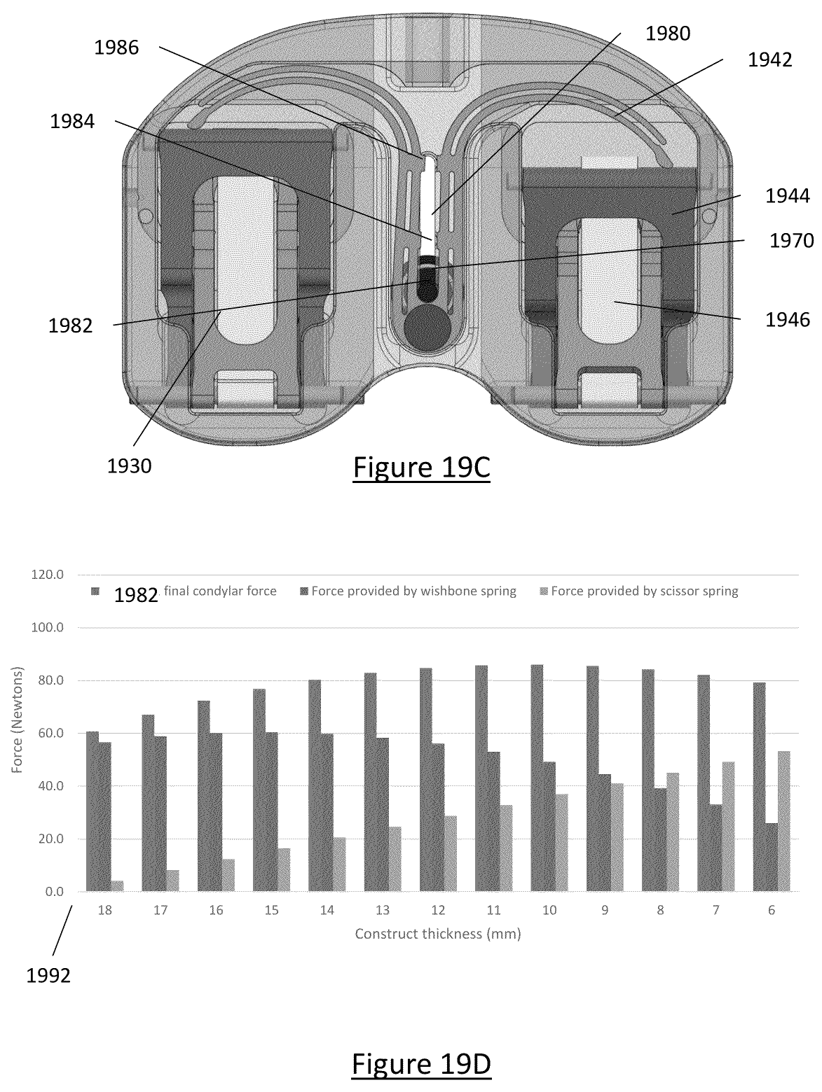

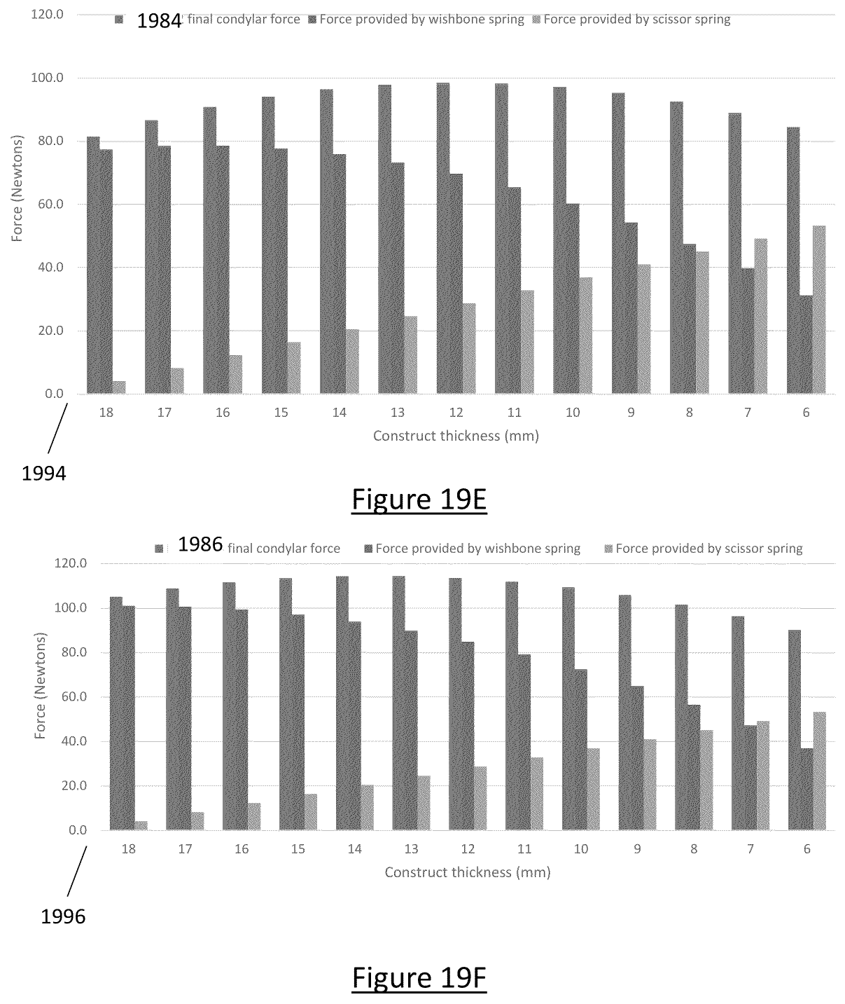

FIGS. 19A, 19B, and 19C show an exemplary embodiment of a ligament balancing device 1900 that is an intraarticular ligament balancing device. FIG. 19A shows a top perspective view, FIG. 19B shows a side perspective view, and FIG. 19C shows a bottom view. In the embodiment shown in FIGS. 19A-19C, the ligament balancing device 1900 includes two separate first plates 1910 and 1920, wherein the first one of the first plate 1910 is positioned to engage with a first condyle of the femur (e.g., either one of the medial condyle or the lateral condyle) and the second one of the first plate 1920 is positioned to engage with the second condyle of the femur (e.g., the other one of either the medial condyle or the lateral condyle). In some embodiments, the exemplary ligament balancing device 1900 includes a second plate 1950 that is configured to engage a tibia. In some embodiments, a first expansion mechanism 1930 is located between the first one of the first plate 1910 and the second plate 1950 and a second expansion mechanism 1940 is located between the second one of the first plate 1920 and the second plate 1950. In some embodiments, the first expansion mechanism 1930 and the second expansion mechanism 1940 are independent from one another. In such embodiments, the range of angular tilt is not linked to (and therefore not limited by) the value of the height. For example, as shown in FIG. 19A, if the first one of the first plate 1910 is in a fully collapsed position with respect to the second plate 1950, then the second one of the first plate 1920 can still independently self-adjust through its entire range of height depending on the ligament laxity. In some embodiments, the first expansion mechanism 1930 and the second expansion mechanism 1940 are each configured so as to provide a range of expansion (e.g., of the thickness of the ligament balancing device 1900 in its most compressed position to the thickness thereof in its most expanded position) of from 6 millimeters to 18 millimeters. In some embodiments, the ligament balancing device 1900 includes a double wishbone spring 1960 that forms part of both the first expansion mechanism 1930 and the second expansion mechanism as will be described hereinafter.

In some embodiments, the second expansion mechanism 1940 includes a wishbone spring portion 1942, an expanding scissor mechanism 1944, and a scissor leaf spring 1946. In some embodiments, the wishbone spring portion 1942 is the portion of the double wishbone spring 1960 that underlies the first plate 1920. In some embodiments, the wishbone spring portion 1942 is positioned so as to apply a force between the second plate 1950 and the expanding scissor mechanism 1944. In some embodiments, the scissor leaf spring 1946 is positioned within the expanding scissor mechanism 1944 so as to apply to a force to the expanding scissor mechanism 1944. In some embodiments, both the wishbone spring portion 1942 and the scissor leaf spring 1946 are configured so as to apply corresponding forces that urge the second one of the first plate 1920 away from the second plate 1950. In some embodiments, the force applied by the wishbone spring portion 1942 (as modified by the varying moment arm of the expanding scissor mechanism 1944) and by the scissor leaf spring 1946 are antagonist to one another, thereby combining to cause the second expansion mechanism 1940 to provide a substantially constant distraction force across the range of expansion of the second expansion mechanism 1940. For brevity, only the elements of the second distraction mechanism 1940 are described herein; the second expansion mechanism 1940 is substantially identical to the first expansion mechanism 1930. In some embodiments, the first expansion mechanism 1930 and the second expansion mechanism 1940 are configured to provide substantially constant distraction forces that are the same as one another. In some embodiments, the first expansion mechanism 1930 and the second expansion mechanism 1940 are configured to provide substantially constant distraction forces that are different from one another. In some embodiments, the ligament balancing device 1900 can be adjusted in situ or as a “back table” adjustment.

In some embodiments, the ligament balancing device 1900 is configured to allow a user (e.g., a surgeon) to adjust the substantially constant distraction force applied by the first expansion mechanism 1930 and the second expansion mechanism 1940 without interchangeable components. In some embodiments, the ligament balancing device 1900 includes an adjustment button 1970 that is configured to be manipulated by a user. In some embodiments, the adjustment button 1970 is movable within an adjustment slot 1980. In some embodiments, the adjustment button 1970 constrains the movement and thereby modifies the length of the effective length double wishbone spring 1960 depending on the position of the adjustment button 1970 within the adjustment slot 1980, thereby modifying the substantially constant distraction force. As shown in FIG. 19C, in some embodiments, the adjustment slot 1980 includes three discrete positions 1982, 1984, and 1986; the adjustment button 1970 is shown positioned in the position 1982 in FIG. 19C. In some embodiments, the locations of the three positions 1982, 1984, and 1986 correspond to three defined values of a distraction force provided by the first expansion mechanism 1930 and the second expansion mechanism 1940 when the adjustment button 1970 is positioned in respective ones of the three discrete positions 1982, 1984, and 1986.

In some embodiments, the position 1982 is positioned such that, when the adjustment button 1970 is positioned in the position 1982, the adjustment button 1970 provides a relatively minimal degree of constraint on the motion of the double wishbone spring 1960. Because this is the case, when the adjustment button 1970 is positioned in the position 1982, the adjustment button adjusts the effective length of the double wishbone spring 1960, thereby configuring the double wishbone spring 1960 so as to cause the first expansion mechanism 1930 and the second expansion mechanism 1940 to each produce a “low” substantially constant distraction force. In some embodiments, the “low” substantially constant distraction force is 80 Newtons. FIG. 19D shows a bar graph 1992 of the distraction force produced by the wishbone spring portion 1942, the distraction force produced by the scissor leaf spring 1946, and the distraction force produced by the second expansion mechanism 1940 (i.e., the sum of the distraction force produced by the wishbone spring portion 1942 and the distraction force produced by the scissor leaf spring 1946) when the adjustment button 1970 is positioned in the position 1982.

In some embodiments, the position 1984 is positioned such that, when the adjustment button 1970 is positioned in the position 1984, the adjustment button 1970 provides a relatively moderate of constraint on the motion of the double wishbone spring 1960. Because this is the case, when the adjustment button 1970 is positioned in the position 1984, the adjustment button adjusts the effective length of the double wishbone spring 1960, thereby configuring the double wishbone spring 1960 so as to cause the first expansion mechanism 1930 and the second expansion mechanism 1940 to each produce a “medium” substantially constant distraction force. In some embodiments, the “medium” substantially constant distraction force is 95 Newtons. FIG. 19E shows a bar graph 1994 of the distraction force produced by the wishbone spring portion 1942, the distraction force produced by the scissor leaf spring 1946, and the distraction force produced by the first expansion mechanism 1930 (i.e., the sum of the distraction force produced by the wishbone spring portion 1942 and the distraction force produced by the scissor leaf spring 1946) when the adjustment button 1970 is positioned in the position 1984.

In some embodiments, the position 1986 is positioned such that, when the adjustment button 1970 is positioned in the position 1986, the adjustment button 1970 provides a relatively high degree of constraint on the motion of the double wishbone spring 1960. Because this is the case, when the adjustment button 1970 is positioned in the position 1986, the adjustment button adjusts the effective length of the double wishbone spring 1960, thereby configuring the double wishbone spring 1960 so as to cause the first expansion mechanism 1930 and the second expansion mechanism 1940 each to produce a “high” substantially constant distraction force. In some embodiments, the “high” substantially constant distraction force is 110 Newtons. FIG. 19F shows a bar graph 1996 of the distraction force produced by the wishbone spring portion 1942, the distraction force produced by the scissor leaf spring 1946, and the distraction force produced by the second expansion mechanism 1940 (i.e., the sum of the distraction force produced by the wishbone spring portion 1942 and the distraction force produced by the scissor leaf spring 1946) when the adjustment button 1970 is positioned in the position 1986.

The specific substantially constant distraction forces discussed above with reference to the ligament balancing device 1900 and the adjustment button 1970 are only exemplary. In other embodiments, the elements of the ligament balancing device 1900 (e.g., the double wishbone spring 1960, the scissor leaf spring 1946, the adjustment slot 1980, the adjustment button 1970, etc.) can be configured to provide a different quantity of substantially constant distraction forces, substantially constant distraction forces that are higher and/or lower in magnitude, larger or smaller increments between substantially constant distraction forces, etc.

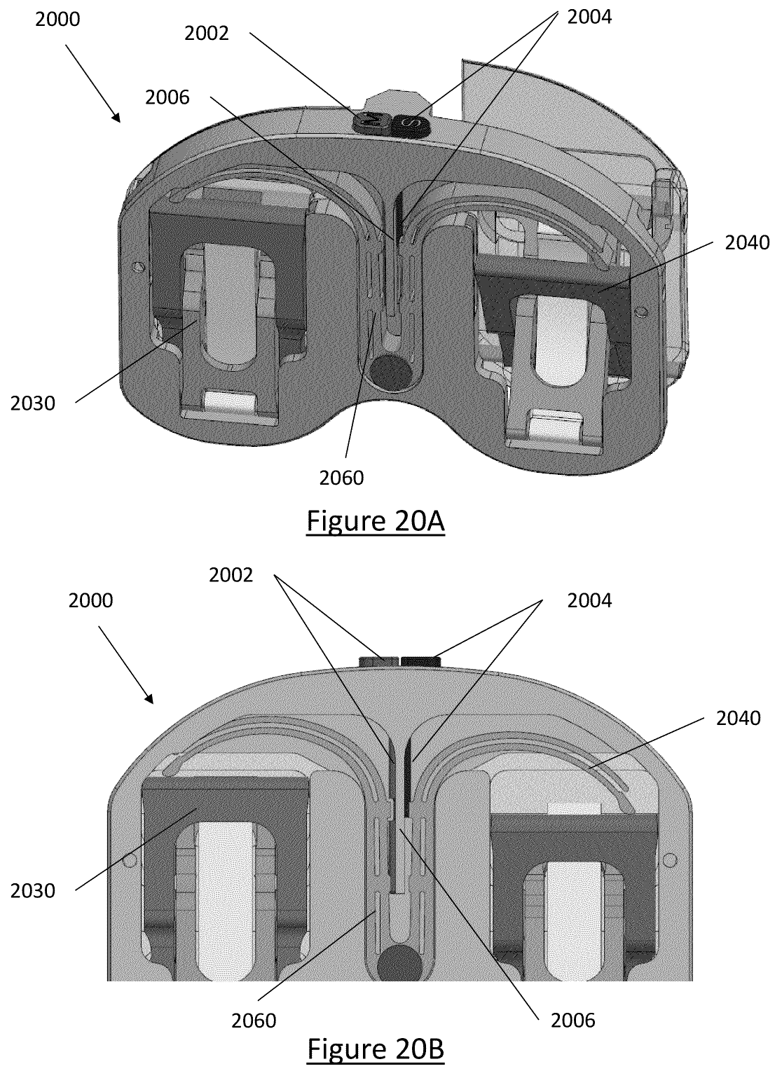

In some embodiments, an exemplary ligament balancing device similar to the exemplary ligament balancing device 1900 is configured to allow a user to separately configure compartment-specific substantially constant distraction forces. FIGS. 20A and 20B show a perspective view and a bottom view, respectively, of such an exemplary ligament balancing device 2000. In some embodiments, the ligament balancing device 2000 is substantially similar to the ligament balancing device 1900 described above with reference to FIG. 19A, other than as described hereinafter. In some embodiments, the ligament balancing device 2000 includes first and second expansion mechanisms 2030 and 2040, respectively, which are substantially similar to the first and second expansion mechanisms 1930 and 1940 of the ligament balancing device 1900. In some embodiments, the ligament balancing device 2000 includes a double wishbone spring 2060 that is substantially similar to the double wishbone spring 1960 of the ligament balancing device 1900. The sides of the double wishbone spring 2060 part of the first and second expansion mechanisms 2030 and 2040 in the same manner as described above with respect to the double wishbone spring 1960 of the ligament balancing device 1900.

In some embodiments, the ligament balancing device 2000 lacks the adjustment button 1970 and the adjustment slot 1980 of the ligament balancing device 1900. Rather, in some embodiments, the ligament balancing device 2000 is configured to accommodate the receipt of interchangeable inserts, and to position such inserts adjacent to the sides of the double wishbone spring 2060 in order to constrain the movement of the double wishbone spring 2060 in a manner similar to that in which the adjustment button 1970 constrains the movement of the double wishbone spring 1960 of the ligament balancing device 1900. FIGS. 20A and 20B show the ligament balancing device 2000 having received a first insert 2002 adjacent to the side of the double wishbone spring 2060 proximate to the first expansion mechanism 2030, and a second insert 2004 adjacent to the side of the double wishbone spring 2060 proximate to the second expansion mechanism 2040. In some embodiments, such as shown in FIGS. 20A and 20B, the ligament balancing device 2000 includes a divider 2006 separating the first insert 2002 from the second insert 2004.

In the embodiment shown in FIGS. 20A and 20B, the first insert 2002 contacts the double wishbone spring 2060 in substantially the same position as the adjustment button 1970 contacts the double wishbone spring 1960 when positioned the position 1984 as shown in FIG. 19C. Thus, the first insert 2002 constrains the portion of the wishbone spring 2060 that forms part of the first expansion mechanism 2030 in the same manner as described above with reference to the position 1984, thereby causing the first expansion mechanism 2030 to produce a “medium” distraction force. As discussed above with reference to the ligament balancing device 1900, in some embodiments, the “medium” distraction force is 95 Newtons, as shown in FIG. 19E. Also in the embodiment shown in FIGS. 20A and 20B, the second insert 2004 contacts the double wishbone spring 2060 in substantially the same position as the adjustment button 1970 contacts the double wishbone spring 1960 when positioned the position 1986 as shown in FIG. 19C. Thus, the second insert 2004 constrains the portion of the wishbone spring 2060 that forms part of the second expansion mechanism 2040 in the same manner as described above with reference to the position 1986, thereby causing the second expansion mechanism 2040 to produce a “high” distraction force. As discussed above with reference to the ligament balancing device 1900, in some embodiments, the “high” distraction force is 110 Newtons, as shown in FIG. 19F. In some embodiments, as an alternative, if the first insert 2002 and/or the second insert 2004 are removed from the ligament balancing device 2000, the motion of the wishbone spring 2060 is not constrained, thereby causing the first expansion mechanism 2030 and/or the second expansion mechanism 2040 to produce a “low” distraction force as described above with reference to the position 1982. As discussed above with reference to the ligament balancing device 1900, in some embodiments, the “high” distraction force is 80 Newtons, as shown in FIG. 19D. In some embodiments, the ligament balancing device 2000 is provided as part of a kit including various inserts such as the insert 2002 and the insert 2004, in order to allow a user to configure the first expansion mechanism 2030 and the second expansion mechanism 2040 each to selectively produce a “low,” “medium,” or “high” distraction force.

The specific substantially constant distraction forces discussed above with reference to the ligament balancing device 2000 and the inserts 2002 and 2004 are only exemplary. In other embodiments, the elements of the ligament balancing device 2000 (e.g., the double wishbone spring 2060, the inserts 2002 and 2004, etc.) can be configured to provide a different quantity of substantially constant distraction forces, substantially constant distraction forces that are higher and/or lower in magnitude, larger or smaller increments between substantially constant distraction forces, etc. In other embodiments, instead of inserts, alternative mechanisms allowing in situ or “back table” adjustments are included. For example, in some embodiments, the substantially constant distraction forces can be adjusted by turning a screw that moves the button 1970, or another similar element, to a selected one of the positions 1982, 1984, or 1986.

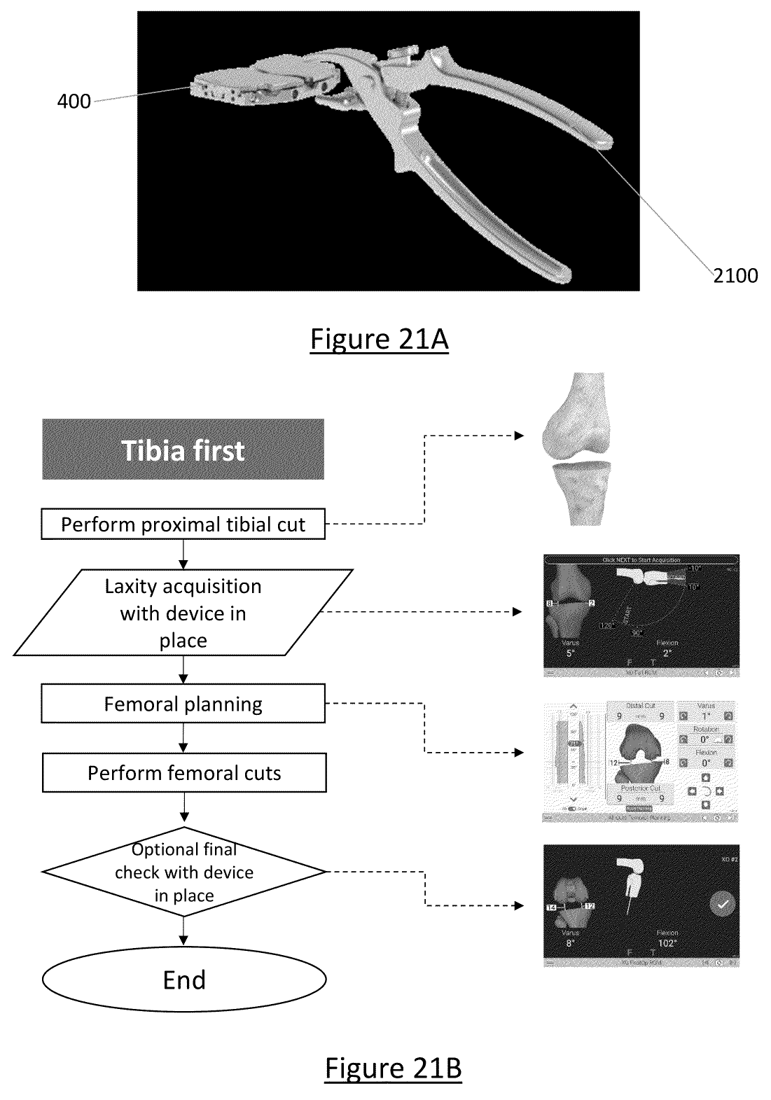

In some embodiments, the exemplary ligament balancing devices 1900 and 2000 provide a user with the ability to adjust an output substantially constant distraction force, while still providing the exemplary ligament balancing device 1900 or 2000 that can be as thin as 6 millimeters when in its most compressed position. In some embodiments, a ligament balancing device 1900 or 2000 that can be fully compressed to a minimum thickness of 6 millimeters provides clinical benefits. For example, in some embodiments, a ligament balancing device 1900 or 2000 that can be compressed to a minimum thickness of 6 millimeters provides for easier usage in patients having tight joint spaces. For example, in cases where a surgeon makes a conservative first bone cut, such as a proximal tibial cut (e.g., in a process as will be described hereinafter with reference to FIGS. 21A and 20B), a resulting joint space (e.g., the space between the femur and the resected tibia) may be about 7 millimeters. In such cases, if a ligament balancing device has a minimum thickness that is greater than the 6 millimeter minimum thickness of the ligament balancing device 1900 or 2000, it may be difficult to determine whether such a device is applying a distraction force to the joint, or is fully compressed and is merely acting as a spacer. Additionally, in such cases, where a ligament balancing device is fully collapsed, it may provide a distraction force greater than it would provide along its range of motion, thereby providing misleading data to a user because the targeted substantially constant distraction force is not being maintained. As such, in some embodiments, the ligament balancing device 1900 or 2000 having a minimum compressed thickness of 6 millimeters provides increased clinical performance for patients with tight joint spaces.

The exemplary embodiments described above incorporate compression springs and leaf springs. In other exemplary embodiments, the expansion mechanisms described herein can also be achieved by any type of spring (e.g., compression springs, leaf springs, extension springs, torsion springs, Belleville springs, drawbar springs, volute springs, garter springs, etc.), manufactured from diverse materials (e.g., metals such as steel or aluminum, elastomeric materials, etc.), and configured in any form and fit to achieve the intended substantially constant distraction force.

In some embodiments, the exemplary ligament balancing devices described above are configured for use in the management of the soft tissue during a total knee arthroplasty type of procedure, wherein the device can be intraarticularly placed into the prepared knee joint and apply a similar or different distraction force on both the lateral compartment and the medial compartment so the surgeon can properly assess the joint space as well as the relative joint alignment under constant distraction force regardless of the joint gap/space of each compartment. In some embodiments, due to the versatility of the disclosed ligament balancing devices, such devices can be provided as part of a conventional mechanical instrumentation set or in combination with a navigation system. Similarly, in some embodiments, the exemplary balancing devices described above can be used at different stages of the procedure regardless of the surgical technique.

In some embodiments, an exemplary ligament balancing device (e.g., the device 400, 900, 1000, 1100, 1300, or 1900) is used in connection with a surgical technique known as “tibia first”. FIG. 21A shows an exemplary ligament balancing device as coupled to an exemplary compression handle 2100 for use in a tibia first technique. FIG. 21B shows the overall surgical workflow of a tibia first technique. In such a technique, the proximal tibial cut is first performed and potential osteophytes around the margins of the native tibia and/or femur are removed. In some embodiments, performance of the proximal tibial cut results in a joint space compatible with the overall dimensions of the exemplary ligament balancing devices in terms of both the transverse dimensions (more or less defined by the perimeter of the proximal tibial cut) and the thickness (defined by the distance between the proximal tibial cut surface and the native femur. Next, in some embodiments, the exemplary ligament balancing device is attached to the compression handle 2100, as shown in FIG. 21A, and is compressed so as to provide the smallest overall thickness of the ligament balancing device (e.g., the lowest distance between the first plate and the second plate). In some embodiments, once compressed, the exemplary ligament balancing device is placed into the joint space and the compression handle is subsequently removed from the ligament balancing device, which results into the application of an axial distraction force to both the medial compartment and the lateral compartment of the joint (e.g., through the medial actuation mechanism located between the medial first plate in contact with the medial condyle of the native femur and the second plate, and through the lateral actuation mechanism located between the lateral first plate in contact with the lateral condyle of the native femur and the second plate, respectively). In some embodiments, at this stage, the surgeon can (1) bring the leg in extension to later balance the knee in extension by recording the joint spaces in extension, and/or (2) bring the leg in flexion to later balance the knee in flexion by recording the joint spaces in flexion, and/or (3) manipulate the leg from extension to flexion to later balance the knee through the arc of motion by recording the joint spaces from extension to flexion, and/or (4) manipulate the leg from flexion to extension to later balance the knee through the arc of motion by recording the joint spaces from flexion to extension. In some embodiments, for any of these options, the joint spaces at specific angles of flexion or through a range of angles of flexion are recorded by the navigation platform as the tracking of the femoral referential (associated with the femoral tracker) into the tibial referential (associated with the tibial tracker). In some embodiments, for options (3) and (4), several methods of handling of the leg are possible, such as placing one hand on the posterior aspect of the femur with the tibia in flexion to prevent the weight of the femur from affecting the measurements, and placing the other hand at the level of the distal tibia or heel with care not to apply a varus/valgus or internal/external rotation moment to the knee joint, while slowly moving the leg from extension to flexion or from flexion to extension. In some embodiments, based on the recorded joint spaces as well as other inputs (e.g., alignment of the leg, size of the knee components, method of alignment), the navigation system computes and displays a femoral plan encompassing the cut parameters, which the surgeon can validate as-is or fine-tune as desired. In some embodiments, once the femoral plan has been confirmed, the femoral cuts are performed under guidance of the navigation system.

In some embodiments, a last optional step includes performing a trial reduction where a trial femoral component is placed onto the prepared femur and the ligament balancing device is placed into the joint space a second time. In some embodiments, by manipulating the leg through the arc of motion, this step offers the possibility of checking the joint gaps and alignment when an axial distraction force is applied to both the medial compartment and the lateral compartment in the same manner as described above.

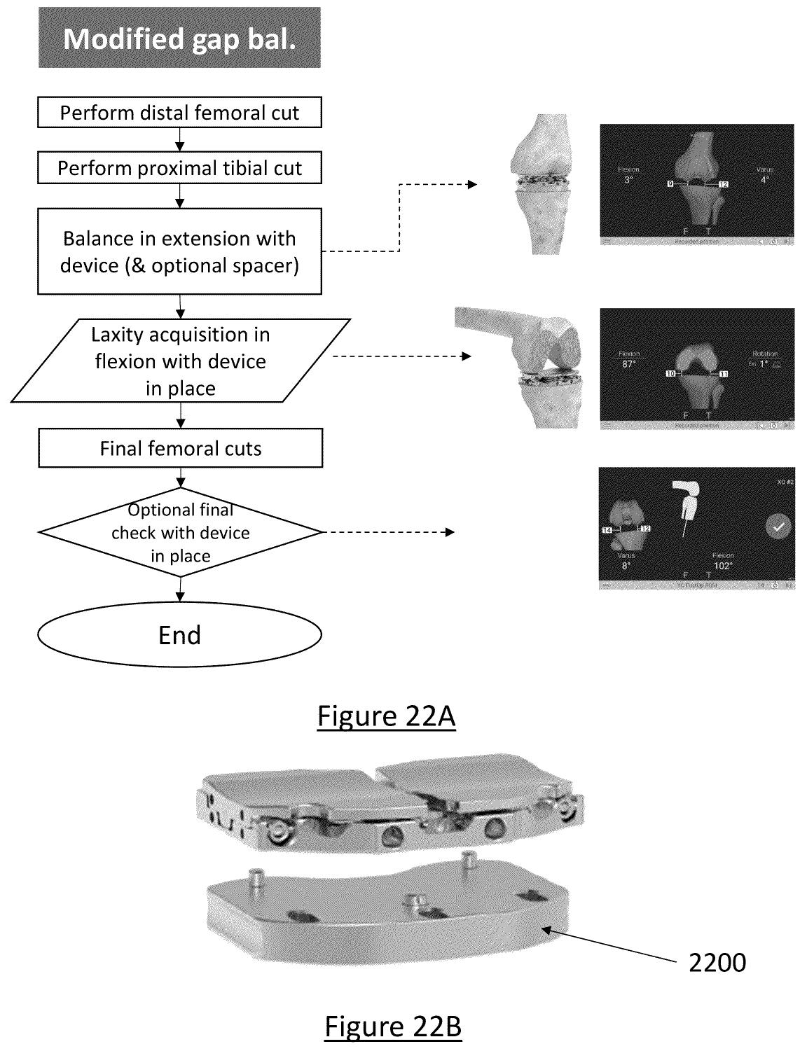

In some embodiments, an exemplary ligament balancing device (e.g., the device 400, 900, 1000, 1100, 1300, or 1900) is used in connection with a surgical technique known as “modified gap balancing”. FIG. 22A describes the overall surgical workflow of a “modified gap balancing” technique. In some embodiments, in a “modified gap balancing” technique, the distal femoral cut is performed first, and the proximal tibial cut is performed second. In some embodiments, in a “modified gap balancing” technique, the proximal tibial cut is performed first and the distal femoral cut is performed second. In some embodiments, performance of both cuts, osteophytes around the margins of the native tibia and/or femur are removed, resulting in a joint space compatible with the overall dimensions of the ligament balancing device in terms of both the transverse dimensions (generally defined by the perimeter of the proximal tibial cut) and thickness (defined by the distance between the proximal tibial cut surface and the distal femoral cut surface). In some embodiments, depending of the gap between the two bone cuts, the axial thickness of the ligament balancing device can be augmented by a spacer 2200 (see FIG. 22B). In some embodiments, the spacer 2200 is positioned distally to the second plate. In some embodiments, the spacer 2200 is positioned proximally to the proximal plate(s). In some embodiments, the ligament balancing device is attached to a compression handle and is compressed to produce the smallest overall thickness of the ligament balancing device (e.g., the lowest distance between the proximal plate(s) and the distal plate). In some embodiments, once assembled to a compression handle (e.g., as shown above in FIG. 21A) and, optionally, to one or more spacers as described above, the compressed ligament balancing device is placed into the joint space while the joint is positioned in extension, and the compression handle is subsequently removed from the ligament balancing device. In some embodiments, such positioning of the exemplary ligament balancing device results in the application of an axial distraction force to both the medial compartment and the lateral compartment of the knee joint. In some embodiments, at this stage, the surgeon assesses the knee in extension by checking the alignment of the overall leg as well as the values of the medial and lateral gaps as displayed on the screen of the navigation system. In some embodiments, based on this information, the surgeon may elect to perform ligament release(s) in order to optimize the alignment. In some embodiments, once proper balance in extension is achieved, the leg is brought into flexion for the assessment of the balance in flexion. In some embodiments, it is necessary to remove the spacer 2200 (if in use to compensate for the thickness of the distal femoral cut) before balancing in flexion because when the knee is in flexion, the proximal plates are in contact with the native posterior condyles of the femur. At this stage, balancing can be performed (1) in a static manner (e.g., at a defined angle associated with the leg being in flexion, typically between 80° and 100° of flexion) or (2) a dynamic manner by bringing the leg from mid-flexion (required to ensure the contact between the still native portion of the femur and the proximal plates) to high flexion or from high flexion to mid-flexion. In some embodiments, the joint spaces at specific angles of flexion or through a range of angles of flexion are recorded by the navigation platform by tracking of the femoral referential (associated with the femoral tracker) and the tibial referential (associated with the tibial tracker). In some embodiments, based on the recorded joint spaces as well as other inputs (e.g., size of the knee components, method of alignment), the navigation system computes and displays a femoral plan encompassing the cut parameters for the final preparation of the femur, which the surgeon can validate as-is or can fine-tune. In some embodiments, once the plan has been confirmed, the final femoral cuts are performed under guidance of the navigation system.

In some embodiments, a last optional step includes performing trial reduction where a trial femoral component is placed onto the prepared femur and the ligament balancing device is placed into the joint space. In some embodiments, by manipulating the leg through the arc of motion, this allows the surgeon to verify the joint gaps and alignment when an axial distraction force is applied to both the medial compartment and the lateral compartment in the same manner as described above.

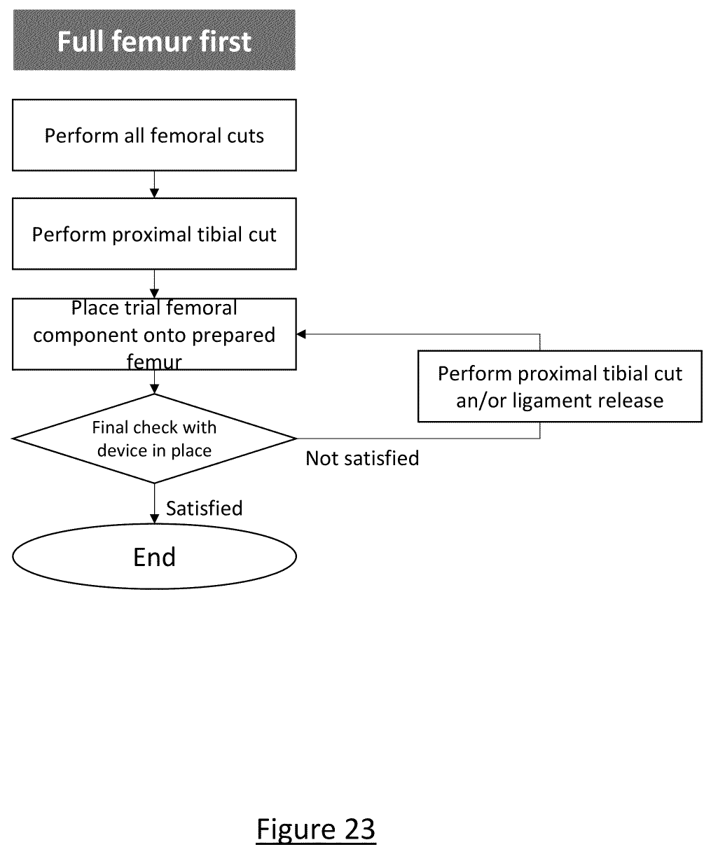

In some embodiments, an exemplary ligament balancing device (e.g., the device 400, 900, 1000, 1100, 1300, or 1900) is used in connection with a surgical technique known as “femur first”. FIG. 23 describes the overall surgical workflow of a “femur first” technique. In some embodiments of a “femur first” technique, all femoral cuts are performed first (e.g., as opposed to the technique shown in FIGS. 22A and 22B, in which only the distal femoral cut is performed first), and the proximal tibial cut is performed second. In some embodiments of a “femur first” technique, the proximal tibial cut is performed first and all femoral cuts are performed second. In some embodiments, following performance of all femoral cuts and the proximal tibial cut, potential osteophytes around the margins of the native tibia and/or femur are removed. In some embodiments, following removal of osteophytes, a trial femoral component is placed on the prepared femur, thereby producing a joint space that is compatible with the overall dimensions of an exemplary ligament balancing device in terms of both the transverse dimensions (generally defined by the perimeter of the proximal tibial cut) and the thickness (defined by the distance between the proximal tibial cut and the trial femoral component). Next, in some embodiments, the ligament balancing device is attached to a compression handle (e.g., as shown above in FIG. 21A) and is compressed to so as to produce the smallest overall thickness of the ligament balancing device (e.g., the lowest distance between the proximal plate(s) and the distal plate). In some embodiments, the compressed ligament balancing device is placed into the joint space and the compression handle is subsequently removed from the ligament balancing device, resulting in the application of an axial distraction force to both the medial compartment and the lateral compartment in the manner described above. In some embodiments, at this point, the surgeon manipulates the leg through the arc of flexion to check the balance and the alignment of the knee by recording these data through the navigation system. In some embodiments, depending on the measured balance and alignment, the surgeon may or may not be satisfied. In some embodiments, if the surgeon is satisfied, the surgeon next implants the final implant components. In some embodiments, if the surgeon is not satisfied, the surgeon may elect to perform subsequent surgical change(s) such as ligament release(s) or bone cut(s) to improve the balance and/or the alignment of the knee joint, and may then reassess the balance and alignment of the knee by manipulating the leg through the arc of flexion after the said surgical change(s).

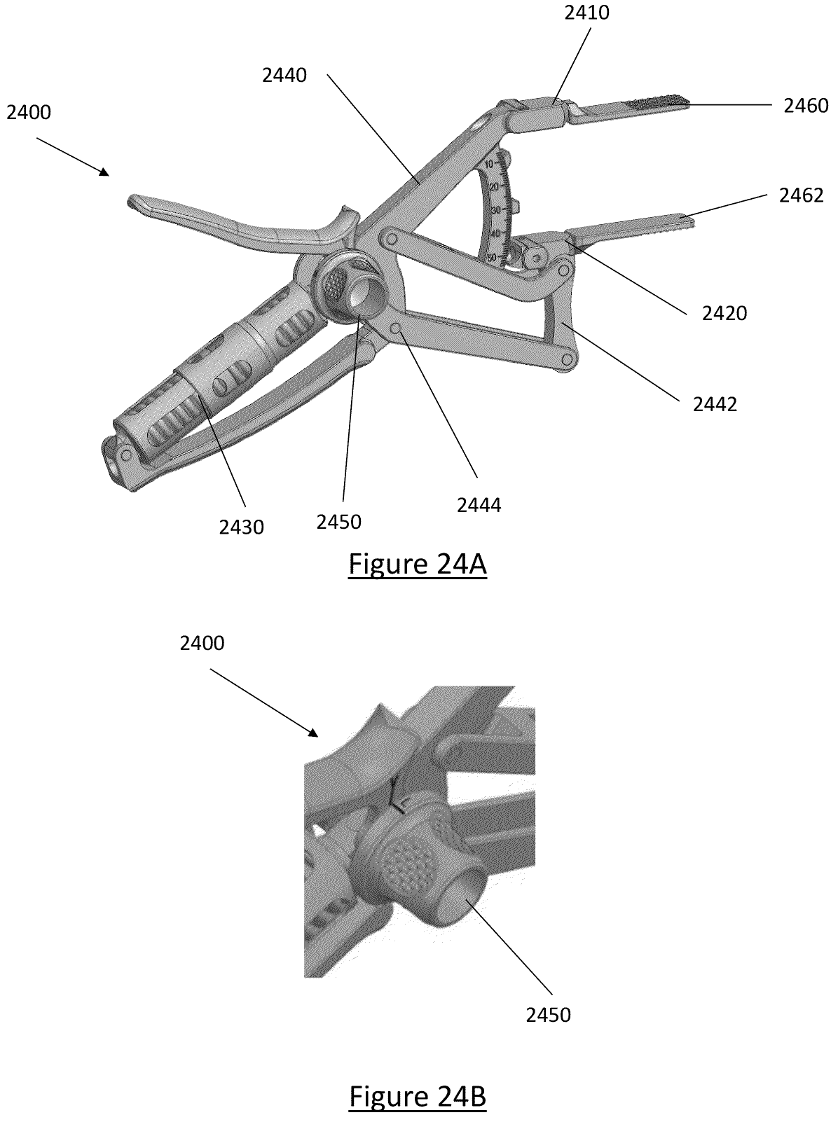

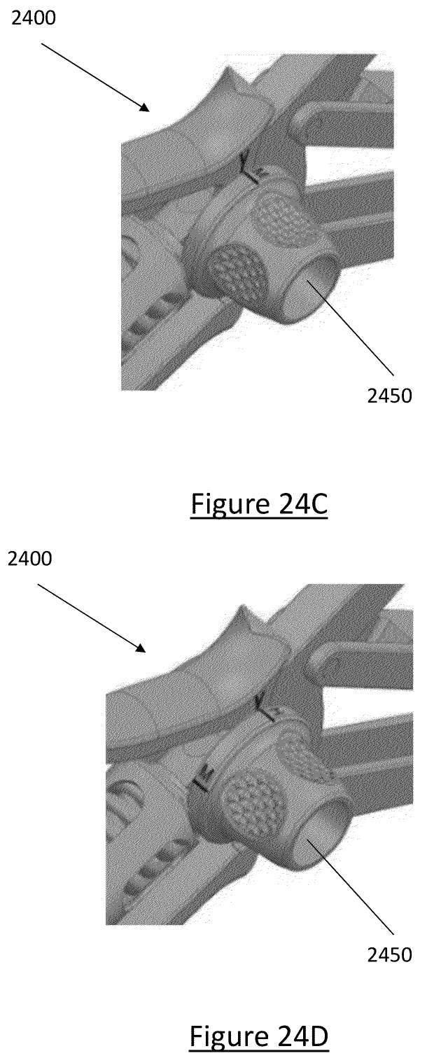

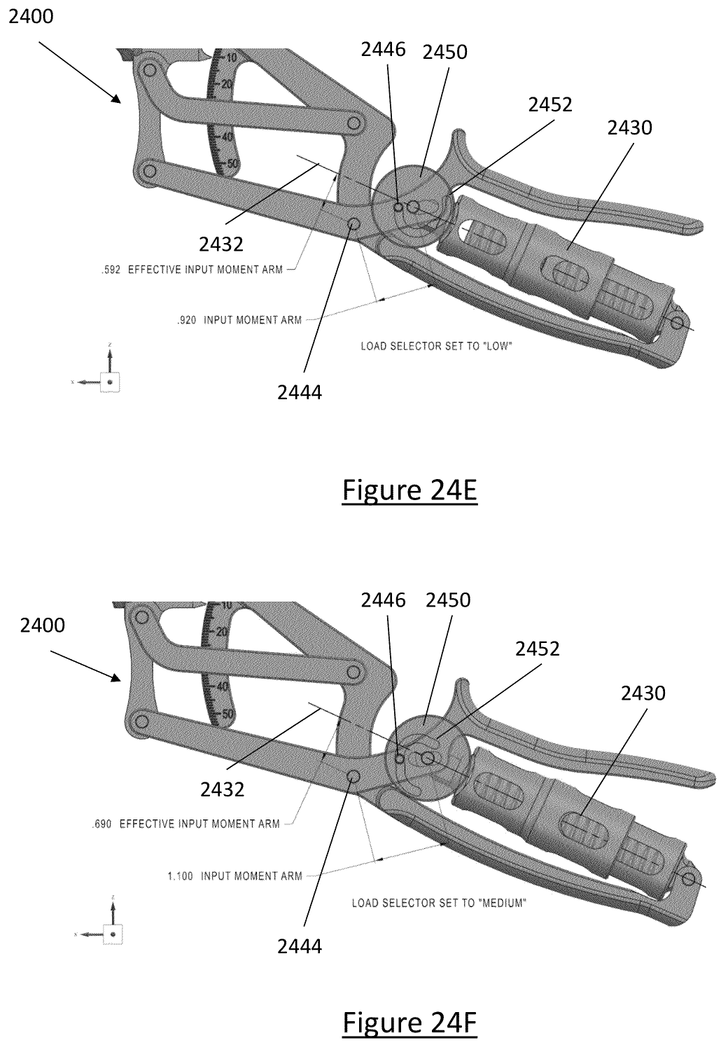

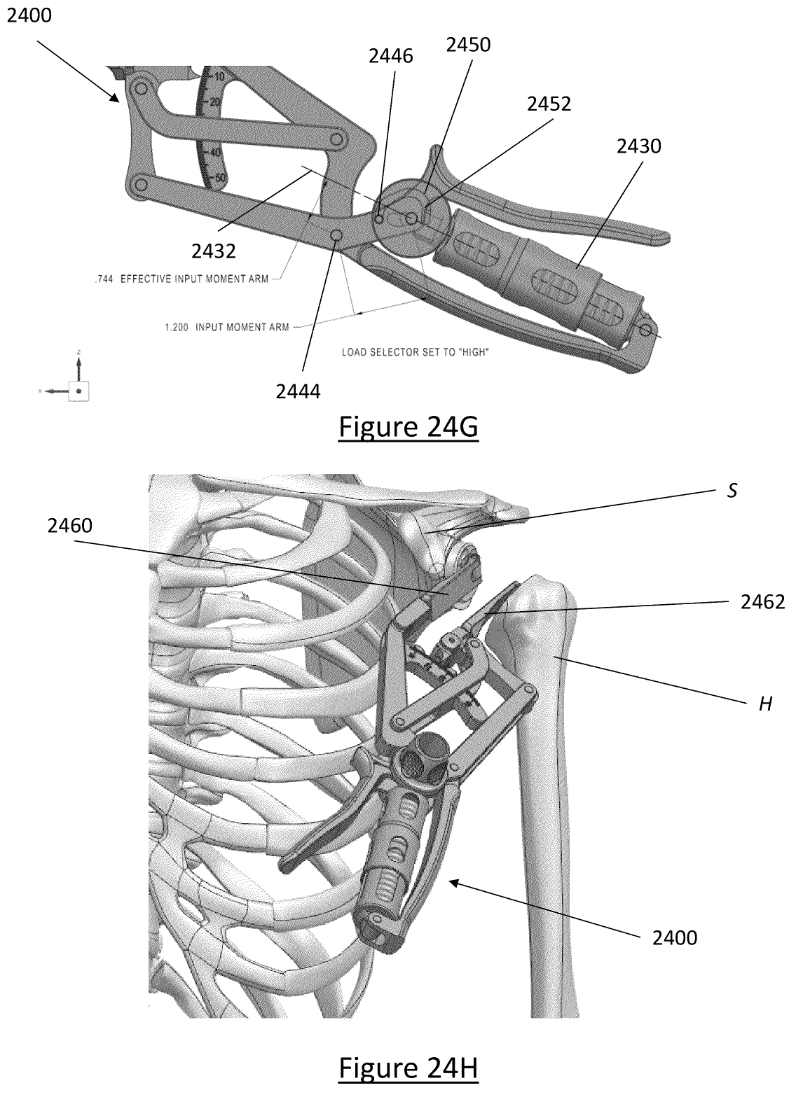

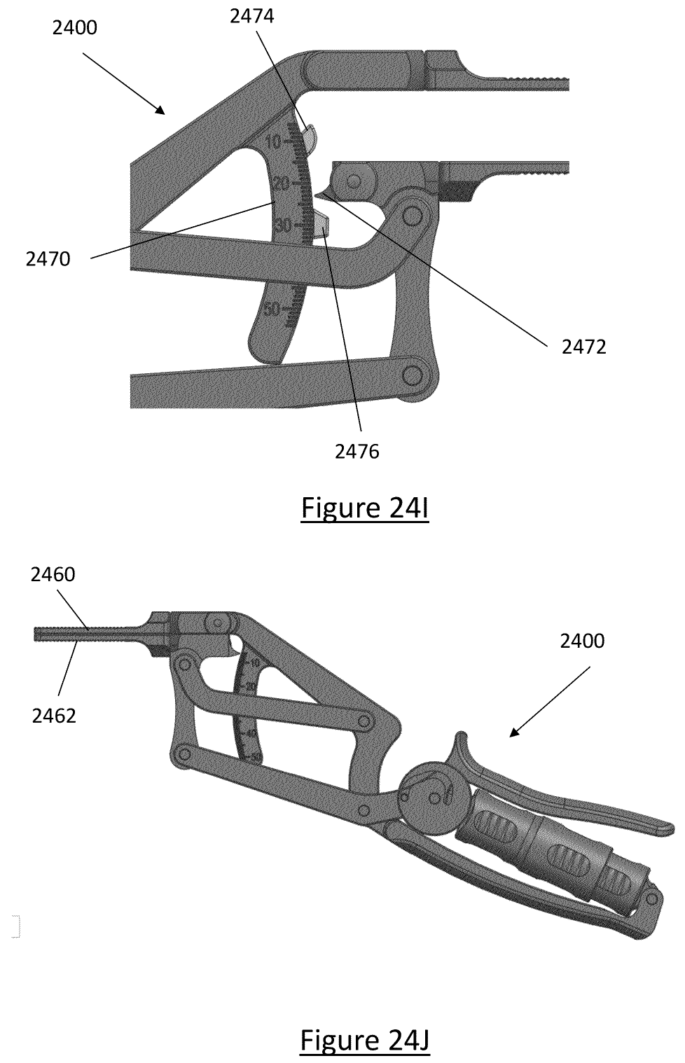

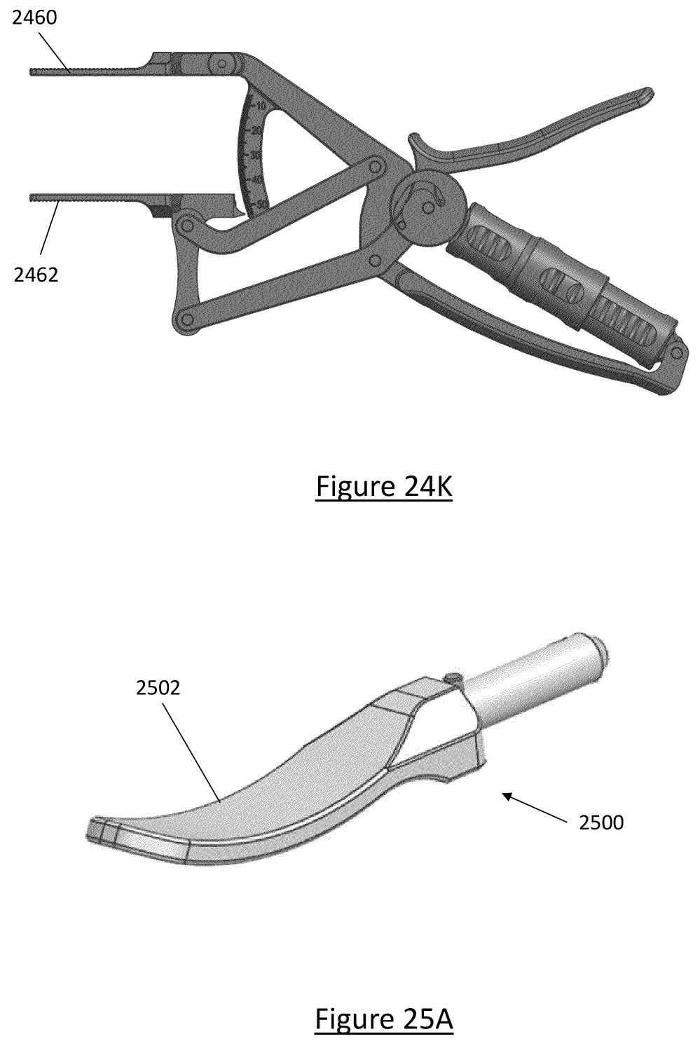

In some embodiments, an extraarticular ligament balancing device is a modular device that is configured to interchangeably receive paddles for use in different surgical procedures and in different joints, e.g., unicondylar joints such as the shoulder or hip. FIG. 24A illustrates a perspective view of such an exemplary ligament balancing device 2400. In some embodiments, the ligament balancing device 2400 includes a first fixation location 2410 and a second fixation location 2420. In some embodiments, the first fixation location 2410 and the second fixation location 2420 include mechanical fixations for securing the position of bone interfacing elements such as described hereinafter. In some embodiments, the mechanical fixations include threaded connections, morse tapers, set screws, or another suitable mechanism for securing such bone interfacing elements in fixed positions and orientations with respect to the ligament balancing device 2400, or in single-degree-of-freedom freely-rotating positions with respect to the ligament balancing device 2400.

In some embodiments, the ligament balancing device 2400 includes at least one mechanical force applying element 2430 (e.g., a compression spring). In some embodiments, the at least one mechanical force applying element 2430 is mechanically coupled to the first fixation location 2410 and to the second fixation location 2420 in a manner so as to urge the first fixation location 2410 and the second fixation location 2420 (and, thereby, bone contacting elements fixed thereto) away from one another. In some embodiments, the ligament balancing device 2400 includes a mechanical linkage 2440 coupling the at least one mechanical force applying element 2430 to the first fixation location 2410 and to the second fixation location 2420. In some embodiments, the mechanical linkage 2440 includes at least one lever arm 2442 and at least one fulcrum 2444. In some embodiments, the mechanical linkage 2440 is configured such that an effective moment arm at which the at least one mechanical force applying element 2430 applies force to urge the first fixation location 2410 away from the second fixation location 2420 varies across the range of expansion between the first fixation location 2410 and the second fixation location 2420. In some embodiments, the force applied by the at least one mechanical force applying element 2430 also varies across the range of expansion between the first fixation location 2410 and the second fixation location 2420. In some embodiments, the varying force applied by the at least one mechanical force applying element 2430 and the varying effective moment arm of the mechanical linkage 2440 combine to produce a substantially constant distraction force between the first fixation location 2410 and the second fixation location 2420 in a manner similar to that described above with reference to other exemplary ligament balancing devices.

In some embodiments, the ligament balancing device 2400 is configured to be adjusted so as to allow a user to adjust the substantially constant distraction force provided by the ligament balancing device 2400. In some embodiments, the ligament balancing device 2400 includes an adjustment dial 2450, as shown in FIGS. 24B-23D. In some embodiments, the adjustment dial 2450 is operable by a user to configure the ligament balancing device 2400 to provide varying values for the substantially constant distraction force. For example, in some embodiments, the adjustment dial 2450 is operable to configure the ligament balancing device 2400 in a selected one of a plurality of discrete configurations. For example, FIG. 24B shows the adjustment dial 2450 positioned so as to configure the ligament balancing device to produce a “low” substantially constant distraction force, FIG. 24C shows the adjustment dial 2450 positioned so as to configure the ligament balancing device to produce a “medium” substantially constant distraction force, and FIG. 24D shows the adjustment dial 2450 positioned so as to configure the ligament balancing device 2400 to produce a “high” substantially constant distraction force. The adjustment dial 2450 shown in FIGS. 24B-23D is configured to allow a user to selectively configure the ligament balancing device 2400 to produce a selected one of three different substantially constant distraction forces, but in other embodiments, the adjustment dial 2450 may be operable to produce other numbers of different substantially constant distraction forces (e.g., two, four, five, six, etc.). In some embodiments, the ligament balancing device 2400 is “right-handed” (e.g., has the adjustment dial 2450 on the right side of the ligament balancing device 2400, as it would be viewed by a surgeon operating the ligament balancing device 2400). In some embodiments, the ligament balancing device 2400 is “left-handed” (e.g., has the adjustment dial 2450 on the left side of the ligament balancing device 2400, as it would be viewed by a surgeon operating the ligament balancing device 2400).

In some embodiments, the adjustment dial 2450 operates to adjust the substantially constant distraction force produced by the ligament balancing device by varying an effective input moment arm within the mechanical linkage 2440 for the force applied by the mechanical force applying element 2430. FIGS. 24E, 24F, and 24G show partial side views of the ligament balancing device 2400 as shown in FIGS. 24B, 24C, and 24D, respectively, illustrating the operation of the adjustment dial 2450. In some embodiments, the adjustment dial 2450 includes a groove 2452 and the mechanical linkage 2440 includes a pin 2446 positioned within the groove 2452. As shown in FIGS. 24E-24G, in some embodiments, the mechanical force applying element 2430 applies a force along an axis 2432. In some embodiments, repositioning of the adjustment dial 2450 repositions the groove 2452 about the pin 2446, thereby relocating the axis 2432 with respect to the fulcrum 2444. In some embodiments, as a result, the effective input moment arm of the force applied by the mechanical force applying element 2430 along the axis 2432 about the fulcrum 2444 is varied. For example, FIG. 24E shows an effective input moment arm of 0.592 inches, FIG. 24F shows an effective input moment arm of 0.690 inches, and FIG. 24G shows an effective input moment arm of 0.744 inches. In some embodiments, increasing the effective input moment arm provides a mechanical advantage to the force applied by the mechanical force applying element 2430, thereby increasing the substantially constant distraction force provided by the ligament balancing device 2400. For example, in the exemplary embodiment shown in FIGS. 24A-G, the “low” setting of the adjustment dial 2450 causes the ligament balancing device 2400 to provide a substantially constant distraction force of 14 pounds, the “medium” setting of the adjustment dial 2450 causes the ligament balancing device 2400 to provide a substantially constant distraction force of 19 pounds, and the “high” setting of the adjustment dial 2450 causes the ligament balancing device 2400 to provide a substantially constant distraction force of 23 pounds. The specific effective input moment arm lengths and substantially constant distraction forces are only exemplary, and in other embodiments, the elements of the ligament balancing device 2400 (e.g., the mechanical force applying element 2430 and/or the mechanical linkage 2440) can be configured to provide a varying quantities of substantially constant distraction forces, substantially constant distraction forces that are higher and/or lower in magnitude, larger or smaller increments between substantially constant distraction forces, etc.

As discussed above, in some embodiments, the ligament balancing device 2400 includes a first fixation location 2410 and a second fixation location 2420 that are configured to interchangeably receive bone-contacting elements (e.g., paddles) that are selected based on the procedure that is being performed. In the embodiment shown in FIG. 24A, the ligament balancing device 2400 is coupled to paddles 2460, 2462 that are configured for use in a shoulder arthroplasty. For example, in some embodiments, the paddle 2460 is configured to contact a patient’s scapula (e.g., at or near a glenoid cavity) and the paddle 2462 is configured to contact a patient’s humeral head or humeral resection surface, thereby to urge the humeral head away from the scapula when the ligament balancing device 2400 is in use. FIG. 24H shows the ligament balancing device 2400 including the paddle 2460 and the paddle 2462 as positioned with reference to the scapula S and humerus H of a representative patient.

In some embodiments, the ligament balancing device 2400 includes an integrated scale for measuring the gap between paddles coupled to the ligament balancing device 2400 (e.g., at varying positions while a joint is moved through a range of motion). In some embodiments, such an integrated scale is mechanically configured to indicate the minimum and maximum gap between the paddles that has been measured during a given time period (e.g., during the time period while a joint receiving the ligament balancing device 2400 is moved through a range of motion). FIG. 24I shows the ligament balancing device 2400 including a scale 2470 and a pointer 2472 indicating the current gap between the paddles.

In some embodiments, the scale 2470 includes markers 2474 and 2476 that are slidably positioned on the scale 2470, with one marker 2474 positioned to one side of the pointer 2472 and the other marker 2476 positioned to the other side of the pointer 2472. In some embodiments, the markers 2474 and 2476 engage the scale 2470 in a manner such that, when the pointer 2472 moves along the scale 2470 in a direction toward one of the markers 2474 or 2476, the pointer 2472 will displace that one of the markers 2474 or 2476 along the scale 2470 by the same amount. In some embodiments, the markers 2474 and 2476 also engage the scale 2470 in a manner such that, when the pointer 2472 moves along the scale 2470 in a direction away from one of the markers 2474 or 2476, the pointer 2472 will not displace that one of the markers 2474 or 2476, but, rather, that one of the markers 2474 or 2476 will remain in the position along the scale 2470 at which the one of the markers 2474 or 2476 had been positioned by motion of the pointer 2472 toward the one of the markers 2474 or 2476 as described above. For example, if the pointer 2472 has moved along the scale 2470 toward the marker 2476 to a position reflecting a gap of 26 millimeters between the paddles coupled to the ligament balancing device 2400, then the marker 2476 will be displaced to a position where the marker 2476 also indicates a gap of 26 millimeters. If the pointer 2472 subsequently moves away from the marker 2476 and toward the marker 2474, the marker 2476 will remain at the position where the marker 2476 indicates a gap of 26 millimeters. In some embodiments, the marker 2474 is positioned to a side of the pointer 2472 that is between the pointer 2472 and the end of the scale 2470 reflecting a minimum gap value, and the marker 2476 is positioned to a side of the pointer 2472 that is between the pointer 2472 and the end of the scale 2470 reflecting a maximum gap value. As such, in some embodiments, motion of the pointer 2472 along the scale 2470 while a joint receiving the ligament balancing device 2400 is moved through a range of motion will cause the marker 2474 to point to a minimum gap observed while the joint is moved through the range of motion, and will cause the marker 2476 to point to a maximum gap observed while the joint is moved through the range of motion. In some embodiments, the scale 2470 is suitable for use during a surgical procedure in which no surgical navigation system or other electronic data capture is utilized.

In some embodiments, the extra-articular actuation mechanism of the ligament balancing device 2400 allows for a larger range of expansion than that which may be provided by an intraarticular ligament balancing device such as those described above. For example, in some embodiments, the ligament balancing device 2400 provides a range of expansion (as measured at the contact surfaces of the connected paddles) of between 6 millimeters in thickness at a most compressed position, and 54 millimeters at a most expanded position. FIG. 24J shows the ligament balancing device 2400 with the paddles 2460 and 2462 as discussed above with reference to FIG. 24A, as positioned in a most compressed state having a distance of 6 millimeters between the respective contact surfaces of the paddles 2460 and 2462. FIG. 24K shows the ligament balancing device 2400 with the paddles 2460 and 2462 as discussed above with reference to FIG. 24A, as positioned in a most expanded position having a distance of 54 millimeters between the respective contact surfaces of the paddles 2460 and 2462.

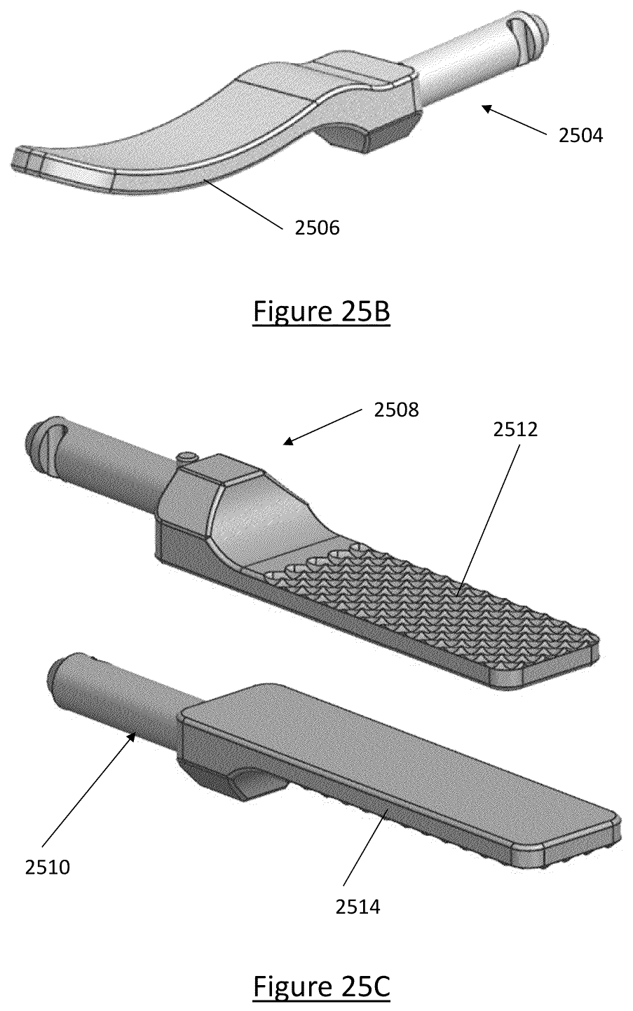

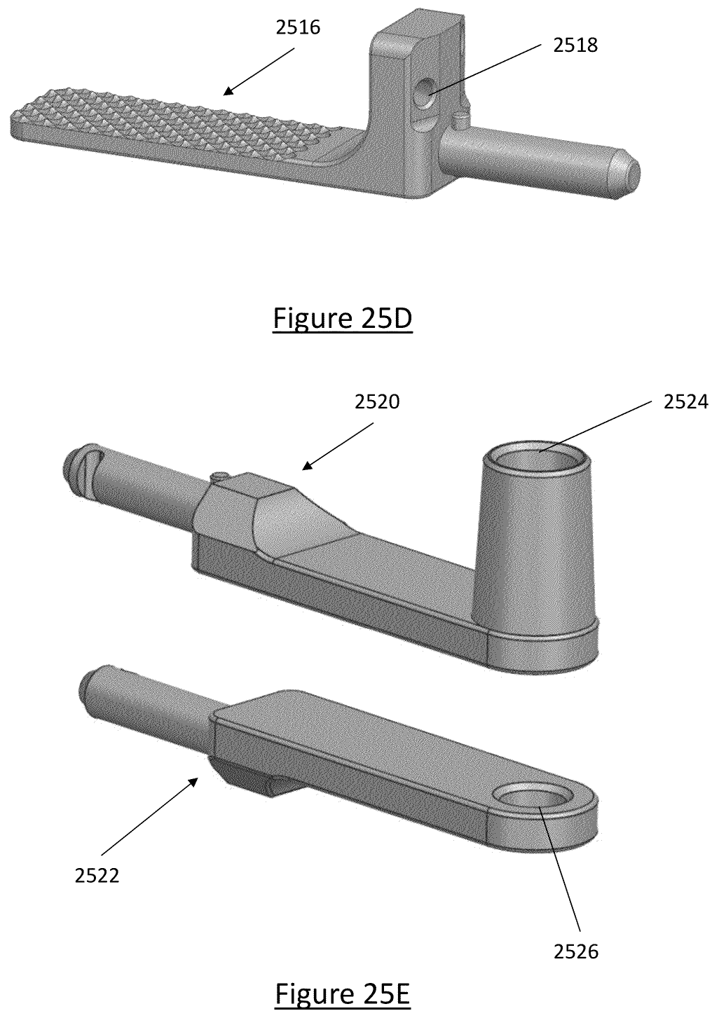

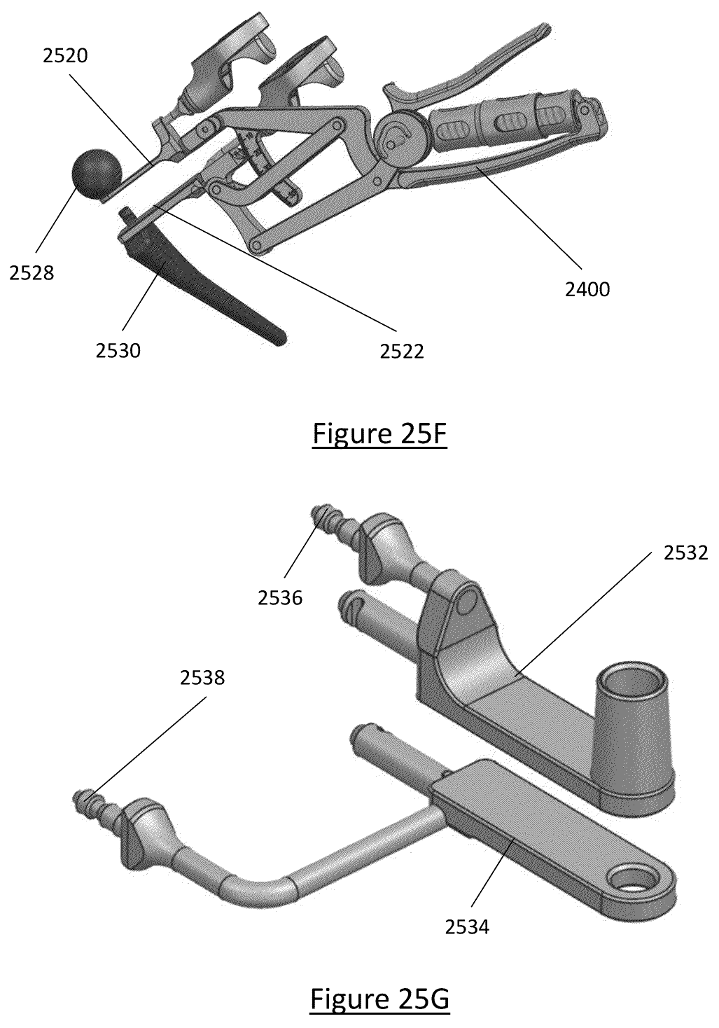

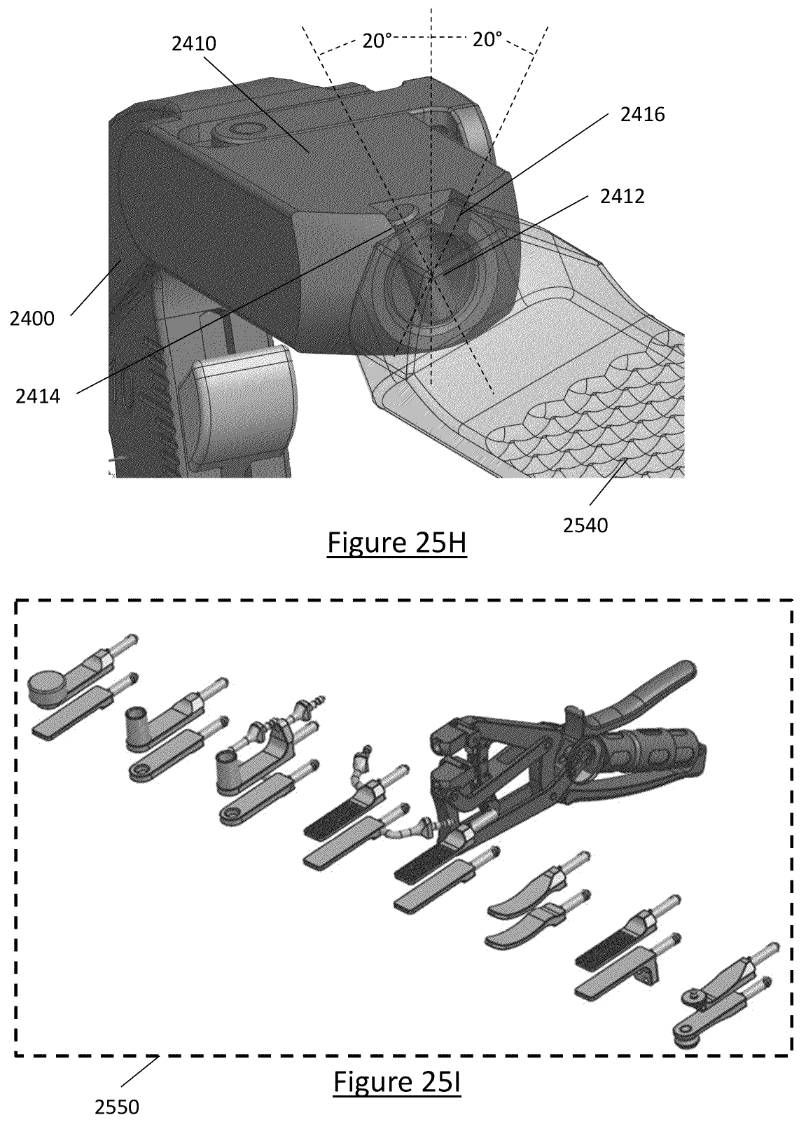

In some embodiments, paddles for use with the ligament balancing device 2400 include a concave contact surface or a convex contact surface configured to contact a bony surface or an instrument in contact with bone. FIG. 25A shows an exemplary paddle 2500 having a concave contact surface 2502. FIG. 25B shows an exemplary paddle 2504 having a convex contact surface 2506.

In some embodiments, paddles for use with the ligament balancing device 2400 include a flat contact surface configured to contact a bony surface or an instrument in contact with bone. FIG. 25C shows exemplary paddles 2508 and 2510 having flat contact surfaces 2512 and 2514, respectively. In some embodiments, such as shown in FIG. 25C, a flat contact surface is textured (e.g., knurled) to prevent slippage.

In some embodiments, paddles for use with the ligament balancing device 2400 include a mechanism to secure the paddle to the receiving bone. For example, FIG. 25D shows an exemplary paddle 2516 having a pin hole 2518 configured to receive a pin to thereby secure the paddle 2516 to a bone. In other embodiments, other securing mechanisms are used.

In some embodiments, paddles for use with the ligament balancing device 2400 are configured to receive instruments from an implant kit that are specific to one or the other side of a joint (e.g., trials, such as a trial femoral head, a trial humeral head, etc.). FIG. 25E shows paddles 2520 and 2522 including fixture points 2524 and 2526, respectively, that are configured to receive instruments from an implant kit. FIG. 25F shows the ligament balancing device 2400 as configured with the paddles 2520 and 2522, and having a trial femoral head 2528 and 2530 coupled to the fixture points 2524 and 2526, respectively.

In some embodiments, paddles for use with the ligament balancing device 2400 include trackers configured to communicate with a surgical navigation system, such as the surgical navigation system commercialized by Exactech, Inc. of Gainesville, Florida under the trade name EXACTECHGPS. FIG. 25G shows paddles 2532 and 2534 having trackers 2536 and 2538, respectively, coupled thereto. The paddles 2532 and 2534 shown in FIG. 25G are structurally similar to the paddles 2520 and 2522 shown in FIG. 25F, but this is only exemplary, and in other embodiments trackers may be included with any other paddles described herein.

In some embodiments, the ligament balancing device 2400 is configured to have paddles fixedly connected thereto (e.g., so as not to have any freedom of independent motion with respect to the ligament balancing device 2400). In some embodiments, the ligament balancing device 2400 is configured to have paddles coupled thereto in a manner so as to have one or more degrees of freedom of independent motion. For example, FIG. 25H shows a connection point between the first fixation location 2410 that is a cylinder 2412. In some embodiments, the cylinder 2412 is rotatably positioned at the first fixation location 2410. As shown in FIG. 25H, a paddle 2540 (shown in a partially transparent view) engages the cylinder. In some embodiments, because the cylinder 2412 is rotatably positioned in the first fixation location 2410, the paddle 2540 that engages the cylinder 2412 is also capable of rotation with respect in a manner so as to allow for axial rotation of the paddle 2540 about the cylinder 2412. In some such embodiments, the first fixation location 2410 is configured to provide a defined degree of freedom of motion to the paddle 2540. In some embodiments, the first fixation location 2410 is configured to allow the paddle 2540 to rotate about a rotation axis that is perpendicular to a direction of expansion between the 2540 and a paddle that is mounted to the second fixation location 2420 (e.g., an axis of expansion of the ligament balancing device 2400). In some embodiments, such as shown in FIG. 25H, the first fixation location includes stops 2414, 2416 positioned so as to constrain the location of the cylinder 2412 to a desired amount, e.g., plus or minus twenty degrees. Possible rotation of a paddle is described above with reference to the first fixation location 2410, but in other embodiments, the same or similar provisions may also be made for the second fixation location 2420.

In some embodiments, a set of paddles are provided in a kit. FIG. 25I illustrates an exemplary kit 2550. For example, in some embodiments, the set of paddles included in a kit are tailored for a specific surgical procedure (e.g., a kit tailored for total shoulder arthroplasty, for total hip arthroplasty, for total knee arthroplasty, for partial knee arthroplasty, etc.). In some embodiments, such a kit includes a selection of the paddles that would be desired by a surgeon performing such a procedure. In some embodiments, a kit includes both paddles including trackers (e.g., of the type shown in FIG. 25G) and paddles excluding trackers. In some embodiments, a kit includes only paddles including trackers, or only paddles excluding trackers. In some embodiments, a kit of paddles is provided together with the ligament balancing device 2400. In some embodiments, a kit of paddles is provided separately from (e.g., without) the ligament balancing device 2400.

According to one example of usage, an exemplary ligament balancing device (e.g., the device 400, 900, 1000, 1100, 1300, 1900, or 2400) is used in conjunction with a surgical navigation system, such as the surgical navigation system commercialized by Exactech, Inc. of Gainesville, Florida under the trade name EXACTECHGPS. In some embodiments, a navigation system includes a display unit combining an infrared charge-coupled device (CCD) camera and a touchscreen tablet intended to be located in the sterile field (under a sterile drape) and directly accessible by the surgeon during the surgery, as well as a set of trackers configured to be rigidly attached to a patient’s bone. In some embodiments, the CCD camera is configured to define the 3D position and orientation of the trackers, surgical instruments, and a system-specific probe within 6 degrees of freedom during the acquisition of anatomical landmarks. In some embodiments, the navigation system includes an intraoperative application configured to compute the acquired data to establish a surgical plan and to provide real-time visual guidance to execute the surgical plan. In some embodiments, the navigation system encompasses a navigated mechanical instrument intended to receive a tracker and facilitate execution of the surgical plan.

In some embodiments, an exemplary ligament balancing device (e.g., the device 400, 900, 1000, 1100, 1300, 1900, or 2400) is used in conjunction with conventional mechanical instrumentation (e.g., in the absence of a navigation system) as a balancer to assess the symmetry of the gaps (e.g., the difference between the medial gap and the lateral gap). In some embodiments, such an assessment can be performed with any of the previously described surgical techniques.

Certain aspects of the exemplary embodiments described above with reference to FIGS. 4A-19F have been described with specific reference to the characteristics of a knee joint. However, the principles embodied by the exemplary embodiments are also applicable to balancing devices adapted for use in other joints.

In some embodiments, aspects of the embodiments described above can be combined with one another so that a surgeon can personalize the ligament balancing device (e.g., a medial module with high stiffness level, a large size, and a concave proximal femoral plate linked with a lateral module with a medium stiffness level, a small size, and a convex proximal femoral plate) depending on the needs of a given patient.

In some embodiments, an exemplary ligament balancing device is configured to maintain a constant or quasi-constant distraction force without including or being coupled to any type of active control arrangement or mechanism. In some embodiments, an exemplary ligament balancing device is configured to maintain a constant or quasi-constant distraction force without including or being coupled to any type of external control mechanism. In some embodiments, an exemplary ligament balancing device is configured to maintain a constant or quasi-constant distraction force without being coupled to any type of external device. In some embodiments, an exemplary ligament balancing device is a self-contained device that is configured to maintain a constant or quasi-constant distraction force without including or being coupled to any type of external control mechanism. In some embodiments, an exemplary ligament balancing device is configured to maintain a constant or quasi-constant distraction force without including any type of powered (e.g., electrically powered) element. In some embodiments, an exemplary ligament balancing device is configured (e.g., sized and shaped) to be positioned intra-articularly and/or intracapsularly within a joint (e.g., to be positioned entirely within the joint space in a manner such that the tissue can be closed with the exemplary ligament balancing device in place. In some embodiments, an exemplary ligament balancing device includes a mechanical actuation mechanism that is positioned entirely within the perimeter of a first plate and the perimeter of a second plate so as to enable the exemplary ligament balancing device to be positioned intra-articularly and/or intracapsularly within a joint.

In some embodiments, the exemplary ligament balancing devices described herein include movable actuation mechanism elements that are positioned so as not to contact soft tissue of a patient’s joint. As a result, the patient’s soft tissue does not adhere to the movable elements, pinch between the movable elements, etc. This provides improved outcomes due to avoiding damage to the soft tissue, and additionally causes the exemplary ligament balancing devices to be easier to clean due to less soft tissue being retained on the device after use has been completed.

While a number of embodiments of the present invention have been described, it is understood that these embodiments are illustrative only, and not restrictive, and that many modifications may become apparent to those of ordinary skill in the art. For example, all dimensions discussed herein are provided as examples only, and are intended to be illustrative and not restrictive.

What is claimed is:

1. A device, comprising:
a first plate configured to interface with a first bone structure of a joint;
a second plate configured to interface with a second bone structure of the joint opposite the first bone structure; and
at least one mechanical actuation mechanism disposed between the first plate and the second plate and configured to apply a distraction force along an axis between the first plate and the second plate so as to urge the first plate and the second plate away from one another, wherein the at least one mechanical actuation mechanism comprises:
a first actuation sub-mechanism, and
a second actuation sub-mechanism,
wherein the first actuation sub-mechanism is configured to provide a first actuation sub-mechanism distraction force, and wherein the second actuation sub-mechanism is configured to provide a second actuation sub-mechanism distraction force that is antagonist to the first actuation sub-mechanism distraction force;
wherein the device is configured so as to have a range of expansion ranging from a minimum distance between the first plate and the second plate to a maximum distance between the first plate and the second plate, and
wherein the first actuation sub-mechanism distraction force and the second actuation sub-mechanism distraction force combine to provide the distraction force that is a substantially constant distraction force across the range of expansion.

2. The device of claim 1, wherein the device is configured so as to allow the substantially constant distraction force to be adjusted.

3. The device of claim 2, wherein the first actuation sub-mechanism comprises an adjustment element that is one of adjustable or interchangeable to thereby adjust the substantially constant distraction force.

4. The device of claim 3, wherein the adjustment element comprises a spring having an adjustable preload.

5. The device of claim 3, wherein the adjustment element comprises an interchangeable cartridge.

6. The device of claim 3, wherein the adjustment element comprises an interchangeable spring.

7. The device of claim 2, wherein the device is configured so as to allow the substantially constant distraction force to be adjusted while positioned in situ.

8. The device of claim 1, wherein the device is configured to apply the substantially constant distraction force to a single condyle of a bicondylar joint.

9. The device of claim 8, wherein the device is configured to be joined to a further one of the device so as to apply the substantially constant distraction force to both condyles of a bicondylar joint.

10. A kit, comprising:
a distraction device, comprising:
a first fixation location configured to receive a first bone contacting element so as to position the first bone contacting element so as to be configured to interface with a first bone structure of a joint;
a second fixation location configured to receive a second bone contacting element so as to position the second bone contacting element so as to be configured to interface with a second bone structure of the joint opposite the first bone structure; and
a mechanical actuation mechanism disposed between the first fixation location and the second fixation location and configured to apply a distraction force along an axis between the first fixation location and the second fixation location so as to urge the first bone contacting element and the second bone contacting element away from one another,
wherein the mechanical actuation mechanism includes a force applying element configured to apply an applied force so as to urge the first bone contacting element and the second bone contacting element away from one another, wherein a magnitude of the applied force varies as the device moves along a range of expansion,
wherein the mechanical actuation mechanism includes a physical parameter that varies as the device moves along the range of expansion, and
wherein the varying applied force and the varying physical parameter combine to cause the distraction force to be a substantially constant distraction force across the range of expansion; and
a plurality of bone contacting elements, wherein the first bone contacting element and the second bone contacting element are selected from among the plurality of bone contacting elements.

11. The kit of claim 10, wherein the device is configured so as to allow the substantially constant distraction force to be adjusted.

12. The kit of claim 11, wherein the varying physical parameter is a varying moment arm.

13. The kit of claim 12, wherein the device comprises an adjustment dial that is adjustable so as to adjust the varying moment arm.

14. The kit of claim 13, wherein the adjustment dial is adjustable to a plurality of discrete settings.

15. The kit of claim 10, wherein the distraction device is configured such that, when in use, the first bone contacting element and the second bone contacting element are positioned in an intraarticular position and the mechanical actuation mechanism is positioned in an extraarticular position.

16. The kit of claim 10, wherein at least one of the plurality of bone contacting elements comprises a trial.

17. The kit of claim 10, wherein at least one of the plurality of bone contacting elements comprises a tracker.

18. The kit of claim 10, wherein at least one of the plurality of bone contacting elements is configured to be secured to bone.

19. The kit of claim 10, wherein at least one of the plurality of bone contacting elements comprises a textured surface.

20. The kit of claim 10, wherein the first fixation location is configured to allow the first bone contacting element to rotate with respect to the first fixation location about an axis that is perpendicular to an axis of expansion of the distraction device.

21. The kit of claim 10, wherein the distraction device comprises a scale and a pointer, and wherein the distraction device is configured such that the pointer indicates a current gap between the first bone contacting element and the second bone contacting element on the scale.

22. The kit of claim 21, wherein the distraction device comprises a marker slidably positioned on the scale, and wherein the pointer is configured to slide the marker along the scale such that the marker indicates one of (1) a maximum gap between the first bone contacting element and the second bone contacting element or (2) a minimum gap between the first bone contacting element and the second bone contacting element.
